(19) United States
(12) Patent Application Publication
(10) Pub. No.: US 2025/0034289 A1
(43) Pub. Date: Jan. 30, 2025

(54) POLYMERIZABLE COMPOSITIONS INCLUDING A POLYMERIZABLE COMPONENT AND A REDOX INITIATION SYSTEM CONTAINING A PHOTOLABILE REDUCING AGENT, AND A PHOTOLABILE REDUCING AGENT

(71) Applicant: 3M INNOVATIVE PROPERTIES COMPANY, St. Paul, MN (US)

(72) Inventors: William H. Moser, Edina, MN (US); Amanda K. Leone, St. Paul, MN (US)

(21) Appl. No.: 18/714,159

(22) PCT Filed: Nov. 22, 2022
(86) PCT No.: PCT/IB2022/061271

§ 371 Date: May 29, 2024

Related U.S. Application Data
(60) Provisional application No. 63/315,683, filed on Mar. 2, 2022.

(51) Int. Cl. C08F 2/50; C07C 49/84; C07D 239/62; C07D 307/62; C07D 407/04

(52) U.S. Cl. CPC C08F 2/50; C07C 49/84; C07D 239/62; C07D 307/62; C07D 407/04; C07C 2601/16

(57) ABSTRACT

Polymerizable compositions are provided, including a polymerizable component and a redox initiation system. The redox initiation system includes a transition metal complex that participates in a redox cycle; an oxidizing agent; and a photolabile reducing agent containing a benzophenone moiety. On exposure to actinic radiation, such as UV, the photolabile reducing agent photolyzes, releasing the reducing agent and initiating the redox-initiated polymerization of the polymerizable component. Also provided is a photolabile reducing agent.

### BACKGROUND

Redox reactions represent an important method for initiating the curing of acrylate, methacrylate, and other vinyl-based resins, including adhesive formulations. Redox-initiated curing often has advantages over photoinitiated curing, including improved depth of cure and a slower accumulation of stress during the initial stages of curing.

A significant challenge in the use of redox initiating systems is finding an optimal balance between stability and reactivity. The reactivity of the redox system needs to be sufficiently high for full curing and attainment of mechanical properties within a short period of time. However, if the reactivity is too great, problems such as premature curing, accumulation of stress, and poor shelf stability of the formulation can be encountered.

Free-radical polymerization of vinyl compound(s) using certain beta-dicarbonyl (i.e., 1,3-dicarbonyl) compounds in the presence of a peroxide and/or oxygen, a halide salt, and a copper compound such as copper acetylacetonate, has been described in U.S. Pat. No. 3,347,954 (Bredereck et al.). Such compositions cause free-radical polymerization of the vinyl compound(s) over time, with shorter times generally being preferred. Since the compositions are spontaneously reactive, it is common practice to provide them as a two-part system such as, for example, a part A and a part B that are combined immediately prior to use.

Another approach has been reported by the Stansbury research group at the University of Colorado (WO 2019/113602 and Kim, K. et al. Macromolecules 2020, S3, 5034). Stansbury has incorporated benzophenone-based ammonium borate salts into one-part acrylic formulations. Irradiation of the formulations is hypothesized to induce cleavage of the salts, generating free radicals which initiate polymerization during the light-curing step and tertiary amine bases which react with peroxides to provide additional free radical redox curing after irradiation has been completed. Although acrylic polymers exhibiting desirable properties have been produced using this “on-demand” curing technique, the highly insoluble nature of the ammonium borate salts represents a significant limitation. To circumvent these solubility challenges, solvents, solubilizing additives, or very specific monomers must be incorporated, thereby greatly reducing the breadth of acrylic formulations which can successfully be utilized with the method.

### SUMMARY

In a first aspect, a polymerizable composition is provided. The polymerizable composition comprises a polymerizable component and a redox initiation system. The redox initiation system comprises a) a transition metal complex that participates in a redox cycle; b) an oxidizing agent; and c) a photolabile reducing agent of the Formula I:

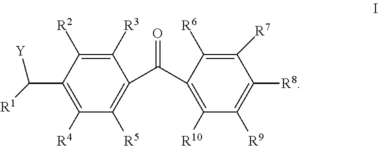

In Formula I, R¹ is of Formula II, Formula III, Formula IV, or Formula V; wherein Y is H or methyl; and wherein each of R², R³, R⁴, R⁵, R⁶, R⁷, R⁸, R⁹, and R¹⁰ is independently H, an alkyl, an alkoxy, or an aryl;

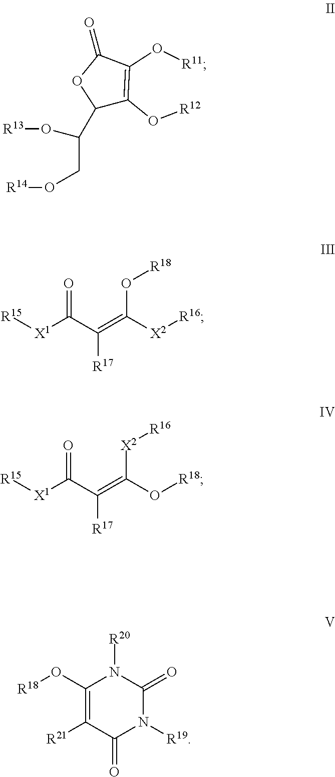

### In Formulas II, III, IV, and V:

R¹² is a covalent bond to Formula I at the R¹ position; R¹¹ is H, an alkyl, an aryl, or a covalent bond to a second group of Formula I at the R¹ position; each of R¹³ and R¹⁴ independently, in combination with the attached O, comprises an alcohol, an alkoxy, an aryloxy, an ester, an ether, a urethane, or a carbonate functional group, or taken together form a 5- or 6-membered ring; each of R¹⁵ and R¹⁶ is independently an optionally substituted 1-18C hydrocarbyl; R¹⁷ is H or an optionally substituted 1-18C hydrocarbyl, wherein R¹⁵+R¹⁶, or R¹⁵+R¹⁷, or R¹⁶+R¹⁷ are optionally taken together to form a 5- or 6-membered ring; R¹⁸ is a covalent bond to Formula I at the R¹ position; each of R¹⁹, R²⁰, and R²¹ is H or an optionally substituted 1-18C hydrocarbyl; and each of X¹ and X² is independently a covalent bond, O, S, —N(R²²)—, —[C(R²²)₂]_{y=1,2,3}—, —CO— or —CO—O—; wherein R²² is H or a 1-18C alkyl.

In a second aspect, a photolabile reducing agent is provided. The photolabile reducing agent is of the Formula I of the first aspect.

Applicants provide a method to overcome the problems mentioned above by creating an “on demand” redox-initiated cure, in which the reducing agent of the redox cure initiator system has latent activity while the formulation is stored and delivered, but then can be triggered when cured properties are required. The redox initiator systems include photolabile reducing agents that contain both a benzophenone moiety and either an ascorbate moiety, diketone moiety, or a barbituric acid moiety.

The present disclosure provides a redox initiator system for initiating polymerization comprising an oxidizing agent, a photolabile reducing agent, and a transition metal complex that participates in a redox cycle. On exposure to actinic radiation, such as UV, the photolabile compound photolyzes, generating the reducing agent and initiating the redox-initiated polymerization. Advantageously, polymerization of the compositions may be initiated by exposure to actinic radiation, but continued irradiation is not required. When the redox initiator system is combined with polymerizable component monomers or oligomers to form a polymerizable composition, the polymerization may be initiated, and then builds molecular weight and physical properties as the composition continues to cure in the absence of light.

Redox initiating systems according to at least certain embodiments of the present disclosure are useful for polymerizing acrylic formulations. Advantageously, the photolabile reducing agents described herein have acceptable solubility in acrylic formulations such that it is not necessary to employ heat, microwave, solvents, solubilizing additives, and/or very specific monomers to incorporate the photolabile reducing agents into acrylic formulations.

In some embodiments, the polymerizable compositions described herein are useful as sealants. In some embodiments, the polymerizable compositions described herein are useful as liquid adhesives, for instance liquid adhesives that are stencil printable. In some embodiments, the polymerizable compositions described herein combine the advantages of PSAs and structural adhesives in the form of a one-part photo-triggered PSA-to-(semi)structural acrylic adhesive. This adhesive acts as a conventional PSA in its uncured or partially cured state, offering easy application, high wet-out, and green strength. The application of a short UV-light trigger initiates a radical-producing redox reaction that continues after the light is removed, inducing a steady rate of cure and a concomitant increase in cohesive strength. Finally, the cure will plateau at a level sufficient to give the adhesive structural or semi-structural performance.

This collection of properties and curing behavior would be especially useful in the common case of a permanent bond between two opaque substrates. In the absence of a UV trigger, the modulus of the adhesive lies below the level dictated by the Dahlquist criterion, meaning the material has tack and it can form a bond to a substrate with only the application of pressure. Next, the UV trigger is applied to the exposed face of the adhesive, initiating the self-sustaining redox reaction but leaving the surface tacky and able to wet out the second substrate within a reasonable period of time (“open time”). After bond closure, the adhesive continues to cure until its modulus reaches a level sufficient for (semi)structural strength.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples may be used in various combinations. In each instance, the recited list serves only as a representative group and should not be interpreted as an exclusive list.

### DETAILED DESCRIPTION

### Glossary

The term “aliphatic” refers to C1-C40, suitably C1-C30, straight or branched chain alkenyl, alkyl, or alkynyl which may or may not be interrupted or substituted by one or more heteroatoms such as O, N, or S. The term “cycloaliphatic” refers to cyclized aliphatic C3-C30, suitably C3-C20, groups and includes those interrupted by one or more heteroatoms such as O, N, or S.

The term “alkyl” refers to a monovalent group that is a radical of an alkane and includes straight-chain, branched, cyclic, and bicyclic alkyl groups, and combinations thereof, including both unsubstituted and substituted alkyl groups. Unless otherwise indicated, the alkyl groups typically contain from 1 to 30 carbon atoms. In some embodiments, the alkyl groups contain 1 to 20 carbon atoms, 1 to 10 carbon atoms, 1 to 6 carbon atoms, 1 to 4 carbon atoms, or 1 to 3 carbon atoms. Examples of “alkyl” groups include, but are not limited to, methyl, ethyl, n-propyl, n-butyl, n-pentyl, isobutyl, t-butyl, isopropyl, n-octyl, n-heptyl, ethylhexyl, cyclopentyl, cyclohexyl, cycloheptyl, adamantyl, norbornyl, and the like.

The term “alkoxy” refers to a monovalent group of formula —ORV where RV is an alkyl as defined above.

The term “alkylene” refers to a divalent group that is a radical of an alkane and includes groups that are linear, branched, cyclic, bicyclic, or a combination thereof. Unless otherwise indicated, the alkylene group typically has 1 to 30 carbon atoms. In some embodiments, the alkylene group has 1 to 20 carbon atoms, 1 to 10 carbon atoms, 1 to 6 carbon atoms, or 1 to 4 carbon atoms. Examples of “alkylene” groups include methylene, ethylene, 1,3-propylene, 1,2-propylene, 1,4-butylene, 1,4-cyclohexylene, and 1,4-cyclohexyldimethylene.

The term “heteroalkylene” refers to a divalent radical of a heteroalkane, which is an alkane having catenary heteroatoms) having at least one catenary O or NH group. Unless otherwise indicated, the heteroalkylene group typically has 1 to 10 carbon atoms, 2 to 10 carbon atoms, 1 to 6 carbon atoms, 1 to 4 carbon atoms, or 1 to 3 carbon atoms, and up to 6 heteroatoms.

Each of “alkenyl” and “ene” refers to a monovalent linear or branched unsaturated aliphatic group with one or more carbon-carbon double bonds, e.g., vinyl.

The term “aromatic” refers to C3-C40, suitably C3-C30, aromatic groups including both carbocyclic aromatic groups as well as heterocyclic aromatic groups containing one or more of the heteroatoms, O, N, or S, and fused ring systems containing one or more of these aromatic groups fused together.

The term “aryl” refers to a monovalent group that is aromatic and, optionally, carbocyclic. The aryl has at least one aromatic ring. Any additional rings can be unsaturated, partially saturated, saturated, or aromatic. Optionally, the aromatic ring can have one or more additional carbocyclic rings that are fused to the aromatic ring. Unless otherwise indicated, the aryl groups typically contain from 6 to 30 carbon atoms. In some embodiments, the aryl groups contain 6 to 20, 6 to 18, 6 to 16, 6 to 12, or 6 to 10 carbon atoms. Examples of an aryl group include phenyl, naphthyl, biphenyl, phenanthryl, and anthracyl.

The term “arylene” refers to a divalent group that is aromatic and, optionally, carbocyclic. The arylene has at least one aromatic ring. Optionally, the aromatic ring can have one or more additional carbocyclic rings that are fused to the aromatic ring. Any additional rings can be unsaturated, partially saturated, or saturated. Unless otherwise specified, arylene groups often have 6 to 20 carbon atoms, 6 to 18 carbon atoms, 6 to 16 carbon atoms, 6 to 12 carbon atoms, or 6 to 10 carbon atoms.

The term “aralkyl” refers to a monovalent group that is an alkyl group substituted with an aryl group (e.g., as in a benzyl group). The term “alkaryl” refers to a monovalent group that is an aryl substituted with an alkyl group (e.g., as in a tolyl group). Unless otherwise indicated, for both groups, the alkyl portion often has 1 to 10 carbon atoms, 1 to 6 carbon atoms, or 1 to 4 carbon atoms and the aryl portion often has 6 to 20 carbon atoms, 6 to 18 carbon atoms, 6 to 16 carbon atoms, 6 to 12 carbon atoms, or 6 to 10 carbon atoms.

The term “aryloxy” refers to a monovalent group that is of formula —OAr where Ar is an aryl group as defined above.

As used herein, the term “ethylenically unsaturated” refers to a group that comprises at least one carbon-carbon double bond, including at least one of (1) a vinyl group (CH2=CH—); (2) a (meth)acryloyloxy group (CH2=CR—(CO)—O—), wherein R is hydrogen or methyl); or (3) a (meth)acrylamido group (CH2=CR—(CO)—NH—), wherein R is hydrogen or methyl; or a maleic group (—(CO)—(CH═CH)—(CO)—).

The term “hydrocarbyl” is inclusive of aryl and alkyl. “Hydrocarbylene” is inclusive of arylene and alkylene.

As used herein, the term “(meth)acrylate” is a shorthand reference to acrylate, methacrylate, or combinations thereof, “(meth)acrylic” is a shorthand reference to acrylic, methacrylic, or combinations thereof, and “(meth)acryl” is a shorthand reference to acryl and methacryl groups. “Acryl” refers to derivatives of acrylic acid, such as acrylates, methacrylates, acrylamides, and methacrylamides. By “(meth)acryl” is meant a monomer or oligomer having at least one acryl or methacryl group, and linked by an aliphatic segment if containing two or more groups. As used herein, “(meth)acrylate-functional compounds” are compounds that include, among other things, a (meth)acrylate moiety.

As used herein, “C1”, “C1”, and “1 carbon” are interchangeable ways of describing a single carbon atom and may be used interchangeably when indicating any number of carbon atoms.

As used herein, the term “actinic radiation” means electromagnetic radiation of wavelength(s) capable of being absorbed by a composition exposed to it and thereby cause at least one chemical reaction or transformation to occur.

As used herein, the term “photoremovable group” means a group that can be removed by exposure to actinic radiation, optionally with one or more subsequent chemical steps.

As used herein, the term “blocked reducing agent” means a compound that does not function as a reducing agent until a blocking group is removed.

As used herein, the term “photolabile reducing agent” means a compound that becomes a reducing agent only once the photoremovable group has been removed.

As used herein, the term “peak wavelength” refers to a single wavelength in which the emission spectrum of a light source achieves its maximum amount.

As used herein, the term “transparent” refers to a material that has at least 50% transmittance, 70% transmittance, or optionally greater than 90% transmittance over at least a 30 nanometer (nm) wavelength band within a particular range of wavelengths and has a thickness of 10 millimeters or less. Suitable ranges of wavelengths include for instance, between 200 nm and 400 nm, between 400 nm and 700 nm, or between 700 nm and 1300 nm.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure.

In this application, terms such as “a”, “an”, and “the” are not intended to refer to only a singular entity, but include the general class of which a specific example may be used for illustration. The terms “a”, “an”, and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise. The term “and/or” means one or all of the listed elements or a combination of any two or more of the listed elements.

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used. Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties). The term “substantially”, unless otherwise specifically defined, means to a high degree of approximation (e.g., within +/−10% for quantifiable properties) but again without requiring absolute precision or a perfect match. Terms such as same, equal, uniform, constant, strictly, and the like, are understood to be within the usual tolerances or measuring error applicable to the particular circumstance rather than requiring absolute precision or a perfect match.

### Polymerizable Compositions

In a first aspect, a polymerizable composition is provided. The polymerizable composition comprises a polymerizable component, and a redox initiation system comprising:
a) a transition metal complex that participates in a redox cycle;
b) an oxidizing agent; and
c) a photolabile reducing agent of the Formula I:

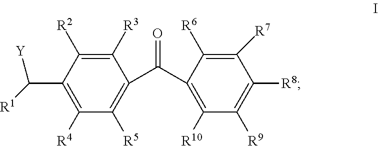

wherein R¹ is of Formula II, Formula III, Formula IV, or Formula V; wherein Y is H or methyl; and wherein each of R², R³, R⁴, R⁵, R⁶, R⁷, R⁸, R⁹, and R¹⁰ is independently H, an alkyl, an alkoxy, or an aryl;

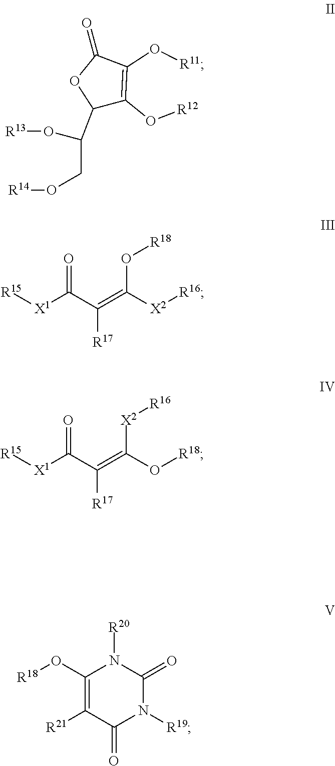

wherein:
R¹² is a covalent bond to Formula I at the R¹ position;
R¹¹ is H, an alkyl, an aryl, or a covalent bond to a second group of Formula I at the R¹ position;
each of R¹³ and R¹⁴ independently, in combination with the attached O, comprises an alcohol, an alkoxy, an aryloxy, an ester, an ether, a urethane, or a carbonate functional group, or taken together form a 5- or 6-membered ring;
each of R¹⁵ and R¹⁶ is independently an optionally substituted 1-18C hydrocarbyl;
R¹⁷ is H or an optionally substituted 1-18C hydrocarbyl, wherein R¹⁵+R¹⁶, or R¹⁵+R¹⁷, or R¹⁶+R¹⁷ are optionally taken together to form a 5- or 6-membered ring;
R¹⁸ is a covalent bond to Formula I at the R¹ position;
each of R⁹, R²⁰, and R²¹ is H or an optionally substituted 1-18C hydrocarbyl; and
each of X¹ and X² is independently a covalent bond, O, S, —N(R²²)—, —[C(R²²)₂]_{y=1,2,3}—, —CO— or —CO—O—; wherein R²² is H or a 1-18C alkyl.

In select embodiments, each of R², R³, R⁴, R⁵, R⁶, R⁷, R⁸, R⁹, and R¹⁰ is H. Accordingly, the benzophenone moiety optionally has a single substitution, located at the position on an aryl ring where R¹ is attached.

The photolabile reducing agent of Formula I includes a benzophenone moiety, which has also been incorporated in polymerizable compositions as a Type II photoinitiator. It has unexpectedly been discovered that the photolabile reducing agent of Formula I can be used in applications that require some amount of open time (e.g., to wet a substrate and form a bond closure between substrates) despite containing the benzophenone moiety. The presence of the benzophenone moiety may provide some initial photoinitiation of a polymerizable composition that may be advantageous in certain cases, such as where the composition would otherwise be experiencing oxygen inhibition.

In Formula I, in some cases R¹ is of Formula II:

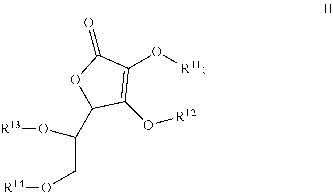

wherein:
R¹² is a covalent bond to Formula I at the R¹ position;
R¹¹ is H, an alkyl, an aryl, or a covalent bond to a second group of Formula I at the R¹ position; and
each of R¹³ and R¹⁴ independently, in combination with the attached O, comprise an alcohol, an alkoxy, an aryloxy, an ester, an ether, a urethane, or a carbonate functional group, or taken together form a 5- or 6-membered ring (e.g., by forming a ketal or acetal group). Optionally, at least one of R¹³ and R¹⁴ comprises a 12-30C alkyl chain in an ester, ether, urethane, or carbonate group to confer solubility and/or miscibility of the reductant in the polymerizable component group.

Some preferred groups include wherein each of R¹¹, R¹³, and R¹⁴ is independently selected from H or an alkyl; R¹³ and R¹⁴ taken together form a 5- or 6-membered ring (e.g., by forming a ketal or acetal group); or R¹⁴ comprises an ester group.

An example of a suitable photolabile reducing agent of Formula I in which R¹ is of Formula II is as follows:

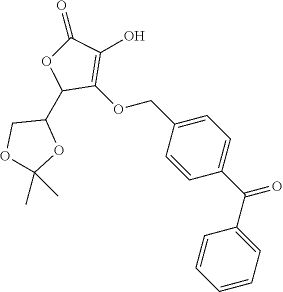 [SMILES: CC1(C)OCC(C2OC(=O)C(O)=C2OCc2ccc(C(=O)c3ccccc3)cc2)O1]

This example may be referred to as benzophenone-blocked ascorbic acid acetonide. In Formula II, R¹¹ is H, R¹² is a covalent bond to Formula I at the R¹ position, and R³ and R⁴ when taken together form a 5-membered ring.

Another example of a suitable photolabile reducing agent of Formula I in which R¹ is of Formula II is as follows:

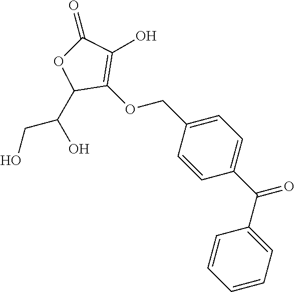 [SMILES: O=C1OC(C(O)CO)C(OCc2ccc(C(=O)c3ccccc3)cc2)=C1O]

This example may be referred to as benzophenone-blocked ascorbic acid. In Formula II, R¹¹ is H, R¹² is a covalent bond to Formula I at the R¹ position, and each of R³ and R⁴ is H.

A further example of a suitable photolabile reducing agent of Formula I in which R¹ is of Formula II is as follows:

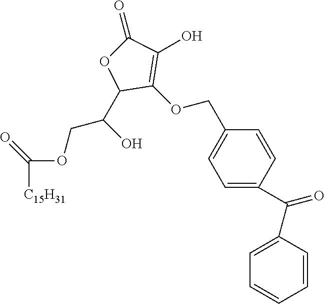

This example may be referred to as benzophenone-blocked ascorbyl palmitate. In Formula II, R¹¹ is H, R¹² is a covalent bond to Formula I at the R¹ position, R¹³ is H, and R¹⁴ in combination with the attached O is an ester group with an attached 15C alkyl chain.

In Formula I, in some cases R¹ is of Formula III or Formula IV:

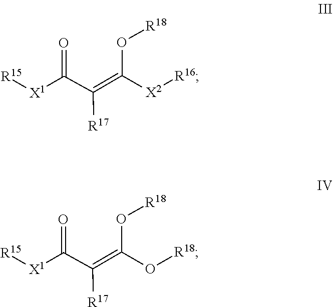

wherein:
each of R¹⁵ and R¹⁶ is independently an optionally substituted 1-18C hydrocarbyl;
R¹⁷ is H or an optionally substituted 1-18C hydrocarbyl, wherein R¹⁵+R¹⁶, or R¹⁵+R¹⁷, or R¹⁶+R¹⁷ are optionally taken together to form a 5- or 6-membered ring;
R¹⁸ is a covalent bond to Formula I at the R¹ position; and
each of X¹ and X² is independently a covalent bond, O, S, —N(R²²)—, —[C(R²²)₂]_{y=1,2,3}-, —CO— or —CO—O—; wherein R²² is H or a 1-18C alkyl.

Some preferred groups include wherein each of R¹⁵ and R¹⁶ is independently an optionally substituted 1-18C hydrocarbyl or taken together form a 5- or 6-membered ring; R¹⁷ is an optionally substituted 1-18C hydrocarbyl; and each of X¹ and X² is a covalent bond.

An example of a suitable photolabile reducing agent of Formula I in which R¹ is of Formula IV is as follows:

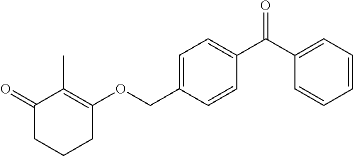 [SMILES: CC1=C(OCc2ccc(C(=O)c3ccccc3)cc2)CCCC1=O]

This example may be referred to as benzophenone-blocked 2-methyl-1,3-cyclohexanedione. In Formula IV, R¹⁵+R¹⁶ are hydrocarbyl groups taken together to form a 6-membered ring, R¹⁷ is a 1C hydrocarbyl (i.e., methyl), R¹⁸ is a covalent bond to Formula I at the R¹ position, and each of X¹ and X² is a covalent bond.

In Formula I, in some cases R¹ is of Formula V:

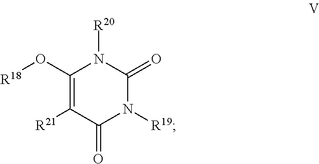

wherein:
R¹⁸ is a covalent bond to Formula I at the R¹ position; and
each of R¹⁹, R²⁰, and R²¹ is independently H or an optionally substituted 1-18C hydrocarbyl.

Some preferred groups include wherein each of R¹⁹ and R²⁰ is independently H, methyl, or benzyl; and R²¹ is an optionally substituted 1-7C hydrocarbyl (e.g., methyl, benzyl, or phenyl).

An example of a suitable photolabile reducing agent of Formula I in which R¹ is of Formula V is as follows:

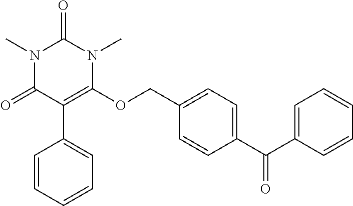 [SMILES: Cn1c(OCc2ccc(C(=O)c3ccccc3)cc2)c(-c2ccccc2)c(=O)n(C)c1=O]

This example may be referred to as benzophenone-blocked 5-phenyl-1,3-dimethylbarbituric acid. In Formula V, R¹⁸ is a covalent bond to Formula I at the R¹ position, each of R¹⁹ and R²⁰ is a 1C hydrocarbyl (i.e., methyl), and R²¹ is a 6C hydrocarbyl (i.e., phenyl).

The redox initiation system comprises a transition metal complex that participates in a redox cycle. Useful transition metal compounds have the general formula: [MLₚ]ⁿ⁺[A^{m-}]_{q}, wherein M is a transition metal selected from groups 5 to 11 in the Periodic Table of the Elements that participates in a redox cycle, L is a ligand, A^{m-} is an anion having a negative charge of m−; m and n are independently integers from 1 to 7 (e.g., 1 to 3); p is an integer from 1 to 9 (e.g., 1 to 2); and q is a whole number selected such that m·q=n.

Typically, M is selected from the group consisting of Cu, Fe, Ru, Cr, Mo, Pd, Ni, Pt, Mn, Rh, Re, Co, V, Au, Nb, and Ag. In certain embodiments, M is Cu. Preferred low valent metals include Cu(II), Fe(II), Ru(II) and Co(II). Other valent states of these same metals may be used, and the active low valent state generated in situ. Optionally, the transition metal complex is a first transition metal complex and the polymerizable composition further comprises a second transition metal complex, in which the M of the first transition metal complex is different than the M of the second transition metal complex.

Useful anions, A⁻, include halides, C₁-C₆ alkoxides, NO₃²⁻, SO₄²⁻, PO₄³⁻, HPO₄²⁻, PF₆⁻, triflate, hexafluorophosphate, methanesulfonate, arylsulfonate, CN, alkyl carboxylates and aryl carboxylates.

When the metal salt is a copper compound, the salt may possess the general formula CuXn, where X is an organic and/or inorganic anion and n=1 or 2. Examples of suitable copper salts include copper chloride, copper acetate, copper acetylacetonate, copper naphthenate, copper salicylate or complexes of copper with thiourea or ethylenediaminetetraacetic acid, and combinations thereof. In some embodiments copper naphthenate is particularly preferred.

The ligand, L, is used to solubilize the transition metal salts in a suitable solvent and adjust the redox potential of the transition metal for appropriate reactivity and selectivity. The ligands can direct the metal complex to undergo the desired one-electron atom transfer process, rather than a two-electron process such as oxidative addition/reductive elimination. The ligands may further enhance the stability of the complexes in the presence of different monomers and solvents or at different temperatures. Acidic monomers and monomers that strongly complex transition metals may still be efficiently polymerized.

Ligands and ligand-metal complexes useful in the initiator systems of the present invention are described in Matyjaszewski and Xia, Chem. Rev., Vol. 101, pp. 2921-2990, 2001.

The molar proportion of photolabile reducing agent (of Formula I) relative to the transition metal complex is generally that which is effective to polymerize the selected polymerizable component(s), but may be from 1000:1 to 5:1, preferably from 500:1 to 25:1, more preferably from 250:1 to 50:1, and most preferably from 200:1 to 75:1. The oxidant and the photolabile reductant of the redox initiator system are used in approximately equimolar amount. Generally, the mole ratio of the oxidant and photolabile reductant is from 1:1.5 to 1.5:1, preferably 1:1.1 to 1.1 to 1.

Suitable oxidizing agents will also be familiar to those skilled in the art, and include but are not limited to persulfuric acid and salts thereof, such as sodium, potassium, ammonium, cesium, and alkyl ammonium salts. Preferred oxidizing agents include peroxides. In select embodiments, the oxidizing agent comprises at least one of an organic peroxide, an organic hydroperoxide, or an inorganic peroxide.

Carbonic peroxyesters are also included among the multifunctional carboxylic acid peroxyesters within the meaning of the present disclosure. Suitable examples include carbonic-diisopropyl-peroxydiester, neodecanoic acid-tertiary-butyl-peroxyester, neodecanoic acid-tertiary-amyl-peroxyester, maleic acid-tertiary-butyl-monoperoxyester, benzoic acid-tertiary-butyl-peroxyester, 2-ethylhexanoic acid-tertiary-butyl-peroxyester, 2-ethylhexanoic acid-tertiary-amyl-peroxyester, carbonic-monoisopropylester-monotertiary-butyl-peroxyester, carbonic-dicyclohexyl-peroxyester, carbonic dimyristyl-peroxyester, carbonic dicetyl peroxyester, carbonic-di(2-ethylhexyl)-peroxyester, carbonic-tertiary-butyl-peroxy-(2-ethylhexyl)ester or 3,5,5-trimethylhexanoic acid-tertiary-butyl-peroxyester, benzoic acid-tertiary-amyl-peroxyester, acetic acid-tertiary-butyl-peroxyester, carbonic-di(4-tertiary-butyl-cyclohexyl)-peroxyester, neodecanoic acid-cumene-peroxyester, pivalic acid-tertiary-amyl-peroxyester and pivalic acid tertiary-butyl-peroxyester.

In particular, carbonic-tertiary-butyl-peroxy-(2-ethylhexyl)ester (commercially available from Arkema, Inc. (King of Prussia, PA) under the trade designation LUPEROX TBEC) or 3,5,5-trimethyl-hexanoic acid-tertiary-butyl-peroxyester (commercially available from Arkema, Inc. (King of Prussia, PA) under the trade designation LUPEROX 270) can be used as organic peroxides according to embodiments of the present disclosure.

Additional suitable organic peroxides include benzoyl peroxides, and hydroperoxides such as cumyl hydroperoxide, t-butyl hydroperoxide, and amyl hydroperoxide.

Exemplary suitable inorganic oxidizing agent include for instance salts of transition metals such as cobalt (III) chloride and ferric chloride, cerium (IV) sulfate, perboric acid and salts thereof, permanganic acid and salts thereof, perphosphoric acid and salts thereof, and mixtures thereof.

The reducing and oxidizing agents are present in amounts sufficient to permit an adequate free-radical reaction rate. This can be evaluated by combining all of the ingredients of the polymerizable composition except for the optional filler, and observing whether or not a hardened mass is obtained.

Preferably, the photolabile reducing agent is present in an amount of at least 0.01 part by weight, and more preferably at least 0.1 part by weight, based on the total weight of the polymerizable (e.g., monomer) components of the polymerizable composition. Preferably, the reducing agent is present in an amount of no greater than 10 parts by weight, and more preferably no greater than 5 parts by weight, based on the total weight of the polymerizable components of the polymerizable composition.

Preferably, the oxidizing agent is present in an amount of at least 0.01 part by weight, and more preferably at least 0.10 parts by weight, based on the total weight of the polymerizable components of the polymerizable composition. Preferably, the oxidizing agent is present in an amount of no greater than 10 parts by weight, and more preferably no greater than 5 parts by weight, based on the total weight of the polymerizable components of the polymerizable composition.

The polymerizable composition optionally further comprises a quatemary ammonium halide or a tertiary ammonium halide that may accelerate the free-radical polymerization rate. In some embodiments, photopolymerizable compositions according to the present disclosure further comprise at least one quaternary ammonium salt. In some embodiments, the at least one quatemary ammonium salt comprises at least one quatemary ammonium salt represented by the formula R₄N⁺X⁻, wherein each R independently represents a hydrocarbyl group having from 1 to 18 carbon atoms and X represents F, Cl, Br, or I. Exemplary R groups include methyl, ethyl, propyl, butyl, hexyl, octyl, decyl, dodecyl, hexadecyl, octadecyl, cyclohexyl, phenyl, benzyl, and phenethyl.

Exemplary quatemary ammonium salts include, tetramethylammonium chloride tetramethylammonium hydroxide, tetramethylammonium bromide, tetramethylammonium hydrogen sulfate, benzyltrimethylammonium chloride, benzyltrimethylammonium bromide, trioctylmethylammonium chloride, benzyltrimethylammonium hydrogen sulfate, benzyltributylammonium chloride, benzyltributylammonium bromide, benzyltributylammonium hydrogen sulfate. Further examples of quaternary ammonium salts are described in U.S. Pat. Nos. 2,740,765 (Parker); 3,437,715 (Da Fano); and 3,840,618 (Da Fano). Combinations of quatemary ammonium salts may also be used. Many quaternary ammonium salts are available commercially.

In some embodiments, the at least one tertiary or quaternary ammonium salt can be at least one tertiary ammonium salt represented by the formula R₃NH⁺X⁻ wherein R and X are as previously defined.

Exemplary tertiary ammonium salts include dibutyl(2-phenylethyl)ammonium chloride, trimethylammonium chloride, trimethylammonium bromide, trimethylammonium iodide, N,N-dimethylethylammonium chloride, and/or N,N-dimethylbenzylammonium chloride.

Typically, if present, the tertiary and/or quatemary ammonium salt(s) is/are present in the free-radically polymerizable composition in an amount of 0.01 to 10 weight percent, preferably 0.1 to 5 weight percent, and more preferably 0.1 to 1 weight percent, based on the total weight of the free-radically polymerizable composition, although other amounts may also be used.

The redox initiator system (i.e., at least the transition metal complex, the oxidizing agent, and the photolabile reducing agent) is present in the polymerizable composition in any amount, but preferably in an amount of from 0.05 to about 10 parts by weight, based on 100 parts by weight of the polymerizable component(s) of the polymerizable composition.

The polymerizable composition comprises a polymerizable component, such as one or more polymerizable components. Often, the polymerizable component comprises at least one ethylenically unsaturated monomer. In select embodiments, the polymerizable component comprises at least one a) vinyl monomer or b) (meth)acrylate monomer or oligomer. Suitable polymerizable component monomers further include (meth)acryloyl monomers (including acrylate esters, amides, and acids) to produce (meth)acrylate homo- and copolymers.

In some embodiments, the polymerizable composition comprises the redox initiator system and one or more vinyl monomers. Vinyl monomers useful in the polymerizable composition include vinyl ethers (e.g. methyl vinyl ether, ethyl vinyl ether), vinyl esters (e.g., vinyl acetate and vinyl propionate), styrene, substituted styrene (e.g., α-methyl styrene), vinyl halide, divinylbenzene, alkenes (e.g., propylene, isomers of butylene, pentene, hexene up to dodecene, isoprene, butadiene) and mixtures thereof.

In some embodiments the polymerizable composition comprises one or more (meth)acrylate ester monomer(s). (Meth)acrylate ester monomers useful in preparing (meth)acrylate (co)polymers are monomeric (meth)acrylic esters of a non-tertiary alcohols, wherein the alcohol contains from 1 to 14 carbon atoms and preferably an average of from 4 to 12 carbon atoms.

Examples of monomers suitable for use as the (meth)acrylate ester monomer include the esters of either acrylic acid or methacrylic acid with non-tertiary alcohols such as ethanol, 1-propanol, 2-propanol, 1-butanol, 2-butanol, 1-pentanol, 2-pentanol, 3-pentanol, 2-methyl-1-butanol, 3-methyl-1-butanol, 1-hexanol, 2-hexanol, 2-methyl-1-pentanol, 3-methyl-1-pentanol, 2-ethyl-1-butanol, 3,5,5-trimethyl-1-hexanol, 3-heptanol, 1-octanol, 2-octanol, isooctyl alcohol, 2-ethyl-1-hexanol, 1-decanol, 2-propylheptanol, 1-dodecanol, 1-tridecanol, 1-tetradecanol, citronellol, dihydrocitronellol, and the like. In some embodiments, the preferred (meth)acrylate ester monomer is the ester of (meth)acrylic acid with butyl alcohol or isooctyl alcohol, or a combination thereof, although combinations of two or more different (meth)acrylate ester monomers are suitable. In some embodiments, the preferred (meth)acrylate ester monomer is the ester of (meth)acrylic acid with an alcohol derived from a renewable source, such as 2-octanol, citronellol, or dihydrocitronellol.

In some embodiments it is desirable for the (meth)acrylic acid ester monomer to include a high glass transition (T_{g}) monomer. The homopolymers of these high T_{g} monomers have a T_{g} of at least 25° C., and preferably at least 50° C. Examples of suitable monomers useful in the present invention include, but are not limited to, t-butyl acrylate, methyl methacrylate, ethyl methacrylate, isopropyl methacrylate, n-butyl methacrylate, isobutyl methacrylate, s-butyl methacrylate, t-butyl methacrylate, stearyl methacrylate, phenyl methacrylate, cyclohexyl methacrylate, isobornyl acrylate, isobornyl methacrylate, benzyl methacrylate, 3,3,5 trimethylcyclohexyl acrylate, cyclohexyl acrylate, N-octyl acrylamide, and propyl methacrylate or combinations thereof.

The (meth)acrylate ester monomer is present in an amount of up to 100 parts by weight, preferably 85 to 99.5 parts by weight based on 100 parts total monomer content used to prepare the polymer, exclusive of the amount of multifunctional (meth)acrylates. Preferably, (meth)acrylate ester monomer is present in an amount of 90 to 95 parts by weight based on 100 parts total monomer content. When high T_{g} monomers are included, the copolymer may include up to 50 parts by weight, preferably up to 20 parts by weight of the (meth)acrylate ester monomer component.

The polymerizable composition may comprise an acid functional monomer, where the acid functional group may be an acid per se, such as a carboxylic acid, or a portion may be a salt thereof, such as an alkali metal carboxylate. Useful acid functional monomers include, but are not limited to, those selected from ethylenically unsaturated carboxylic acids, ethylenically unsaturated sulfonic acids, ethylenically unsaturated phosphonic or phosphoric acids, and mixtures thereof. Examples of such compounds include those selected from acrylic acid, methacrylic acid, itaconic acid, fumaric acid, crotonic acid, citraconic acid, maleic acid, oleic acid, β-carboxyethyl (meth)acrylate, 2-sulfoethyl methacrylate, styrene sulfonic acid, 2-acrylamido-2-methylpropanesulfonic acid, vinylphosphonic acid, and mixtures thereof.

Due to their availability, acid functional monomers of the acid functional copolymer are generally selected from ethylenically unsaturated carboxylic acids, i.e. (meth)acrylic acids. When even stronger acids are desired, acidic monomers include the ethylenically unsaturated sulfonic acids, ethylenically unsaturated phosphoric acids, and ethylenically unsaturated phosphonic acids. The acid functional monomer is generally used in amounts of 0.5 to 15 parts by weight, preferably 1 to 15 parts by weight, most preferably 2 to 8 parts by weight, based on 100 parts by weight total monomer.

The polymerizable composition may comprise a polar monomer. The polar monomers useful in preparing the copolymer are both somewhat oil soluble and water soluble, resulting in a distribution of the polar monomer between the aqueous and oil phases in an emulsion polymerization. As used herein the term “polar monomers” are exclusive of acid functional monomers.

Representative examples of suitable polar monomers include but are not limited to 2-hydroxyethyl (meth)acrylate; N-vinylpyrrolidone; N-vinylcaprolactam; acrylamide; mono- or di-N-alkyl substituted acrylamide; t-butyl acrylamide; dimethylaminoethyl acrylamide; N-octyl acrylamide; tetrahydrofurfuiryl (meth)acrylate, poly(alkoxyalkyl) (meth)acrylates including 2-(2-ethoxyethoxy)ethyl (meth)acrylate, 2-ethoxyethyl (meth)acrylate, 2-methoxyethoxyethyl (meth)acrylate, 2-methoxyethyl methacrylate, polyethylene glycol mono(meth)acrylates; alkyl vinyl ethers, including vinyl methyl ether; and mixtures thereof. Preferred polar monomers include those selected from the group consisting of tetrahydrofurfuryl (meth)acrylate, 2-hydroxyethyl (meth)acrylate and N-vinylpyrrolidone. The polar monomer may be present in amounts of 0 to 10 parts by weight, preferably 0.5 to 5 parts by weight, based on 100 parts by weight total monomer.

The polymerizable composition may further comprise a vinyl monomer when preparing acrylic copolymers. When used, vinyl monomers useful in the (meth)acrylate polymer include vinyl esters (e.g., vinyl acetate and vinyl propionate), styrene, substituted styrene (e.g., α-methyl styrene), vinyl halide, divinylbenzene, and mixtures thereof. As used herein vinyl monomers are exclusive of acid functional monomers, acrylate ester monomers and polar monomers. Such vinyl monomers are generally used at 0 to 5 parts by weight, preferably 1 to 5 parts by weight, based on 100 parts by weight total monomer when preparing acrylic copolymers.

A multifunctional (meth)acrylate may be incorporated into the blend of polymerizable monomers. Examples of useful multifunctional (meth)acrylates include, but are not limited to, di(meth)acrylates, ti(meth)acrylates, and tetra(meth)acrylates, such as 1,6-hexanediol di(meth)acrylate, poly(ethylene glycol) di(meth)acrylates, polybutadiene di(meth)acrylate, polyurethane di(meth)acrylates, and propoxylated glycerin tri(meth)acrylate, and mixtures thereof. The amount and identity of multifunctional (meth)acrylate is tailored depending upon application of the adhesive composition, for example, adhesives, or hardcoats.

Examples of suitable (meth)acrylates and (meth)acrylamides include mono-, di-, and poly-(meth)acrylates and (meth)acrylamides such as, for example, 1,2,4-butanetriol tri(meth)acrylate, 1,3-butylene glycol di(meth)acrylate, 1,3-propanediol di(meth)acrylate, 1,4-butanediol di(meth)acrylate, 1,4-cyclohexanediol di(meth)acrylate, 1,6-hexanediol di(meth)acrylate, 1,6-hexanediol monomethacrylate monoacrylate, 2-phenoxyethyl (meth)acrylate, alkoxylated cyclohexanedimethanol di(meth)acrylates, alkoxylated hexanediol di(meth)acrylate, alkoxylated neopentyl glycol di(meth)acrylate, allyl (meth)acrylate, bis[1-(2(meth)acryloxy)]-p-ethoxyphenyldimethylmethane, bis[1-(3-(meth)acryloxy-2-hydroxy)]-p-propoxyphenyldimethylmethane, caprolactone-modified dipentaerythritol hexa(meth)acrylate, caprolactone modified neopentyl glycol hydroxypivalate di(meth)acrylate, cyclohexanedimethanol di(meth)acrylate, diethylene glycol di(meth)acrylate, dipentaerythritol penta(meth)acrylate, dipropylene glycol di(meth)acrylate, ditrimethylolpropane tetra(meth)acrylate, ethoxylated (10) bisphenol A di(meth)acrylate, ethoxylated (20) trimethylolpropane tri(meth)acrylate, ethoxylated (3) bisphenol A di(meth)acrylate, ethoxylated (3) trimethylolpropane tri(meth)acrylate, ethoxylated (30) bisphenol A di(meth)acrylate, ethoxylated (4) bisphenol A di(meth)acrylate, ethoxylated (4) pentaerythritol tetra(meth)acrylate, ethoxylated (6) trimethylolpropane tri(meth)acrylate, ethoxylated (9) trimethylolpropane tri(meth)acrylate, ethoxylated bisphenol A di(meth)acrylate, ethyl (meth)acrylate, ethylene glycol di(meth)acrylate, 2-ethylhexyl (meth)acrylate, glycerol tri(meth)acrylate, hydroxypivalaldehyde-modified trimethylolpropane di(meth)acrylate, octyl (meth)acrylate, nonyl (meth)acrylate, decyl (meth)acrylate, lauryl (meth)acrylate, tridecyl (meth)acrylate, isobornyl (meth)acrylate, isopropyl (meth)acrylate, methyl (meth)acrylate, neopentyl glycol di(meth)acrylate, n-hexyl (meth)acrylate, pentaerythritol tetra(meth)acrylate, pentaerythritol tri(meth)acrylate, polyethylene glycol (200) di(meth)acrylate, polyethylene glycol (400) di(meth)acrylate, polyethylene glycol (600) di(meth)acrylate, propoxylated (3) glyceryl tri(meth)acrylate, propoxylated (3) trimethylolpropane tri(meth)acrylate, propoxylated (5.5) glyceryl tri(meth)acrylate, propoxylated (6) trimethylolpropane tri(meth)acrylate), propoxylated neopentyl glycol di(meth)acrylate, sorbitol hexa(meth)acrylate, stearyl (meth)acrylate, tetraethylene glycol di(meth)acrylate, tetrahydrofurfuryl (meth)acrylate, tricyclodecanedimethanol di(meth)acrylate, triethylene glycol di(meth)acrylate, trimethylolpropane tri(meth)acrylate, tripropylene glycol di(meth)acrylate, tris(2-hydroxyethyl)isocyanurate tri(meth)acrylate, (meth)acrylamide, N,N-dimethylacrylamide, N-vinylpyrrolidone, N-vinylcaprolactam, methylene bis(meth)acrylamide, diacetone (meth)acrylamide, (meth)acryloylmorpholine, urethane (meth)acrylates, polyester (meth)acrylates, epoxy (meth)acrylates, copolymerizable mixtures of (meth)acrylated monomers such as those in U.S. Pat. No. 4,652,274 (Boettcher et al.), (meth)acrylated oligomers such as those of U.S. Pat. No. 4,642,126 (Zador et al.), poly(ethylenically-unsaturated) carbamoyl isocyanurates such as those disclosed in U.S. Pat. No. 4,648,843 (Mitra), and combinations thereof.

Suitable urethane (meth)acrylate oligomer(s) may include aromatic urethane acrylates, aliphatic urethane acrylates, aromatic/aliphatic urethane acrylates and combinations thereof. Many urethane (meth)acrylate oligomer(s) are available commercially. Suitable examples of urethane (meth)acrylate oligomer(s) may be obtained from Arkema, King of Prussia, Pennsylvania, and marketed as CN1964 (aliphatic urethane dimethacrylate), CN1968 (low viscosity urethane methacrylate oligomer), CN310 (urethane acrylate oligomer), CN996 (aromatic polyester-based urethane diacrylate oligomer); SOLTECH LTD., Yangsan, South Korea, and marketed as SUA5371 (difunctional aliphatic urethane acrylate oligomer); Nippon Soda Co. Ltd., Chiyoda, Japan, and marketed as TE-2000 (polybutadiene urethane methacrylate), TEAI-1000 (polybutadiene urethane acrylate); Dymax, Torrington, Connecticut, and marketed as BR-3747AE (aliphatic polyether urethane acrylate), BRC-843S (hydrophobic urethane acrylate), BR640D (polybutadiene urethane acrylate), and combinations thereof. Other suitable urethane (meth)acrylate oligomer(s) may be prepared by the reaction of (i) a polyisocyanate and a hydroxy-functional (meth)acrylate, and/or (ii) a polyisocyanate, a polyol, and a hydroxy-functional (meth)acrylate. In some examples, the urethane (meth)acrylate is a reaction product of one or more polyisocyanate(s), one or more polyol(s), and one or more hydroxy-functional (meth)acrylate(s).

Typically, the multifunctional (meth)acrylate is present in amounts up to 100 parts, preferably 0.1 to 100 parts, based 100 parts by weight of remaining polymerizable monofunctional monomers. In some embodiments the multifunctional (meth)acrylate is used in amounts of greater than 50 parts by weight, based on the 100 parts by weight of remaining polymerizable monomers. In some embodiments, the multifunctional (meth)acrylate may be present in amounts from 0.01 to 35 parts, preferably 0.05 to 10 parts, based on 100 parts total monomers of the polymerizable composition for adhesive applications, and greater amounts for hardcoats.

In such embodiments, an acrylic copolymer may be prepared from a polymerizable composition comprising:
i) up to 100 parts by weight, preferably 85 to 99.5 parts by weight of an (meth)acrylic acid ester;
ii) 0 to 15 parts by weight, preferably 0.5 to 15 parts by weight of an acid functional ethylenically unsaturated monomer;
iii) 0 to 15 parts by weight of a non-acid functional, ethylenically unsaturated polar monomer;
iv) 0 to 5 parts by weight vinyl monomer;
v) 0 to 100 parts by weight of a multifunctional (meth)acrylate, preferably 50 to 100 parts by weight, relative to i-iv; and
vi) the redox initiator system (including the complex, oxidant and photolabile reductant) in amounts from about 0.1 weight percent to about 5.0 weight percent, relative to 100 parts total monomer i-v.

The polymerizable composition may also include other additives. Examples of suitable additives include tackifiers (e.g., rosin esters, terpenes, phenols, and aliphatic, aromatic, or mixtures of aliphatic and aromatic synthetic hydrocarbon resins), surfactants, plasticizers (other than physical blowing agents), nucleating agents (e.g., talc, silica, or TiO₂), pigments, dyes, reinforcing agents, solid fillers, stabilizers (e.g., UV stabilizers), and combinations thereof. The additives may be added in amounts sufficient to obtain the desired properties for the cured composition being produced. The desired properties are largely dictated by the intended application of the resultant polymeric article.

Adjuvants may optionally be added to the compositions such as colorants, abrasive granules, antioxidant stabilizers, thermal degradation stabilizers, light stabilizers, conductive particles, flow agents, film-forming polymers, bodying agents, flatting agents, inert fillers, binders, blowing agents, fungicides, bactericides, surfactants, plasticizers, rubber tougheners and other additives known to those skilled in the art. They also can be substantially unreactive, such as fillers, both inorganic and organic. These adjuvants, if present, are added in an amount effective for their intended purpose.

In some embodiments, a toughening agent may be used. The toughening agents which are useful in the present invention are polymeric compounds having both a rubbery phase and a thermoplastic phase such as: graft polymers having a polymerized, diene, rubbery core and a polyacrylate, polymethacrylate shell; graft polymers having a rubbery, polyacrylate core with a polyacrylate or polymethacrylate shell; and elastomeric particles polymerized in situ in the epoxide from free radical polymerizable monomers and a copolymerizable polymeric stabilizer.

Examples of useful toughening agents of the first type include graft copolymers having a polymerized, diene, rubbery backbone or core to which is grafted a shell of an acrylic acid ester or methacrylic acid ester, monovinyl aromatic hydrocarbon, or a mixture thereof, such as disclosed in U.S. Pat. No. 3,496,250 (Czerwinski), incorporated herein by reference. Preferable rubbery backbones comprise polymerized butadiene or a polymerized mixture of butadiene and styrene. Preferable shells comprising polymerized methacrylic acid esters are lower alkyl (C1-C4) substituted methacrylates. Preferable monovinyl aromatic hydrocarbons are styrene, alphamethylstyrene, vinyltoluene, vinylxylene, ethylvinylbenzene, isopropylstyrene, chlorostyrene, dichlorostyrene, and ethylchlorostyrene. It is important that the graft copolymer contain no functional groups that would poison the catalyst.

Examples of useful toughening agents of the second type are acrylate core-shell graft copolymers wherein the core or backbone is a polyacrylate polymer having a glass transition temperature below about 0° C., such as polybutyl acrylate or polyisooctyl acrylate to which is grafted a polymethacrylate polymer (shell) having a glass transition above about 25° C., such as polymethylmethacrylate.

The third class of toughening agents useful in the invention comprises elastomeric particles that have a glass transition temperature (T) below about 25° C. before mixing with the other components of the composition. These elastomeric particles are polymerized from free radical polymerizable monomers and a copolymerizable polymeric stabilizer that is soluble in the resins. The free radical polymerizable monomers are ethylenically unsaturated monomers or diisocyanates combined with coreactive difunctional hydrogen compounds such as diols, diamines, and alkanolamines.

Useful toughening agents include core/shell polymers such as methacrylate-butadiene-styrene (MBS) copolymer wherein the core is crosslinked styrene/butadiene rubber and the shell is polymethylacrylate (for example, ACRYLOID KM653 and KM680, available from Rohm and Haas, Philadelphia, PA), those having a core comprising polybutadiene and a shell comprising poly(methyl methacrylate) (for example, KANE ACE M511, M521, BIIA, B22, B31, and M901 available from Kaneka Corporation, Houston, TX and CLEARSTRENGTH C223 available from ATOFINA, Philadelphia, PA), those having a polysiloxane core and a polyacrylate shell (for example, CLEARSTRENGTH S-2001 available from ATOFINA and GENIOPERL P22 available from Wacker-Chemie GmbH, Wacker Silicones, Munich, Germany), those having a polyacrylate core and a poly(methyl methacrylate) shell (for example, PARALOID EXL2330 available from Rohm and Haas and STAPHYLOID AC3355 and AC3395 available from Takeda Chemical Company, Osaka, Japan), those having an MBS core and a poly(methyl methacrylate) shell (for example, PARALOID EXL2691A, EXL2691, and EXL2655 available from Rohm and Haas) and the like and mixtures thereof. Preferred modifiers include the above-listed ACRYLOID and PARALOID modifiers and the like, and mixtures thereof.

The toughening agent is useful in an amount equal to about 1-35 parts by weight, preferably about 3-25 parts by weight, relative to 100 parts by weight of the polymerizable component of the polymerizable composition. The toughening agent adds strength to the composition after curing without reacting with the component of the polymerizable composition or interfering with curing.

In some embodiments the polymerizable composition may include one or more non-free radically polymerizable film-forming polymers. The term “film-forming organic polymer” refers to an organic polymer that will uniformly coalesce upon drying. Film-forming polymers suitable for use in the compositions are generally thermoplastic organic polymers. Examples of suitable polymers include: polyesters, for example, polyethylene terephthalate or polycaprolactone; copolyesters, for example, polyethylene terephthalate isophthalate; polyamides, for example, polyhexamethylene adipamide; vinyl polymers, for example, poly(vinyl acetate/methyl acrylate), poly(vinylidene chloride/vinyl acetate); polyolefins, for example, polystyrene and copolymers of styrene with acrylate(s) such as, for example, poly(styrene-co-butyl acrylate); polydienes, for example, poly(butadiene/styrene); acrylic polymers, for example, poly(methyl methacrylate-co-ethyl acrylate), poly(methyl acrylate-co-acrylic acid); polyurethanes, for example, reaction products of aliphatic, cycloaliphatic or aromatic diisocyanates with polyester glycols or polyether glycols; and cellulosic derivatives, for example, cellulose ethers such as ethyl cellulose and cellulose esters such as cellulose acetate/butyrate. Combinations of film-forming polymers may also be used. Methods and materials for preparing aqueous emulsions or latexes of such polymers are well known, and many are widely available from commercial sources.

In some embodiments the polymerizable composition may include at least one filler. In some embodiments the total amount of filler is at most 50 wt. %, preferably at most 30 wt. %, and more preferably at most 10 wt. % filler. Fillers may be selected from one or more of a wide variety of materials, as known in the art, and include organic and inorganic filler. Inorganic filler particles include silica, submicron silica, zirconia, submicron zirconia, and non-vitreous microparticles of the type described in U.S. Pat. No. 4,503,169 (Randklev).

Filler components include nanosized silica particles, nanosized metal oxide particles, and combinations thereof. Nanofillers are also described in U.S. Pat. Nos. 7,090,721 (Craig et al.), 7,090,722 (Budd et al.), 7,156,911(Kangas et al.), and 7,649,029 (Kolb et al.).

In some embodiments the filler may be surface modified. A variety of conventional methods are available for modifying the surface of nanoparticles including, e.g., adding a surface-modifying agent to nanoparticles (e.g., in the form of a powder or a colloidal dispersion) and allowing the surface-modifying agent to react with the nanoparticles. Other useful surface-modification processes are described in, e.g., U.S. Pat. No. 2,801,185 (Iler), U.S. Pat. No. 4,522,958 (Das et al.) U.S. Pat. No. 6,586,483 (Kolb et al.), each incorporated herein by reference.

Surface-modifying groups may be derived from surface-modifying agents. Schematically, surface-modifying agents can be represented by the formula X-Y, where the X group is capable of attaching to the surface of the particle (i.e., the silanol groups of a silica particle) and the Y group is a reactive or non-reactive functional group. A non-functional group does not react with other components in the system (e.g., a substrate). Non-reactive functional groups can be selected to render the particle relatively more polar, relatively less polar or relatively non-polar. In some embodiments the non-reactive functional group “Y” is a hydrophilic group such as an acid group (including carboxylate, sulfonate and phosphonate groups), ammonium group or poly(oxyethylene) group, or hydroxyl group. In other embodiments, “Y” may be a reactive functional group such as an ethylenically unsaturated polymerizable group, including vinyl, allyl, vinyloxy, allyloxy, and (meth)acryloyl, that may be free-radically polymerized with the polymerizable resin or monomers.

Such optional surface-modifying agents may be used in amounts such that 0 to 100%, generally 1 to 90% (if present) of the surface functional groups (Si—OH groups) of the silica nanoparticles are functionalized. The number of functional groups is experimentally determined where quantities of nanoparticles are reacted with an excess of surface modifying agent so that all available reactive sites are functionalized with a surface modifying agent. Lower percentages of functionalization may then be calculated from the result. Generally, the amount of surface modifying agent is used in amount sufficient to provide up to twice the equal weight of surface modifying agent relative to the weight of inorganic nanoparticles. When used, the weight ratio of surface modifying agent to inorganic nanoparticles is preferably 2:1 to 1:10. If surface-modified silica nanoparticles are desired, it is preferred to modify the nanoparticles prior to incorporation into the coating composition.

The present polymerizable compositions are also useful in the preparation of hardcoats and structural or semi-structural adhesives. The term “hardcoat” or “hardcoat layer” means a layer or coating that is located on the external surface of an object, where the layer or coating has been designed to at least protect the object from abrasion.

The present disclosure provides hardcoat compositions comprising the redox initiator system and a multifunctional (meth)acrylate monomer comprising two (preferably three) or more (meth)acrylate groups, and/or a multifunctional (meth)acrylate oligomer and optionally a (meth)acrylate-functional diluent.

Molecular weight may be controlled through the use of chain transfer agents and chain retarding agents, including mercaptans, disulfides, triethyl silane, carbon tetrabromide, carbon tetrachloride, alpha-methyl styrene and others such as are known in the art.

The present polymerization may be conducted in bulk, or in a solvent. Solvents, preferably organic, can be used to assist in the dissolution of the initiator and initiator system in the polymerizable monomers, and as a processing aid. Preferably, such solvents are not reactive with components. It may be advantageous to prepare a concentrated solution of the transition metal complex in a small amount of solvent to simplify the preparation of the polymerizable composition.

Suitable solvents include ethers such as diethyl ether, ethyl propyl ether, dipropyl ether, methyl t-butyl ether, di-t-butyl ether, glyme (dimethoxyethane), diglyme, diethylene glycol dimethyl ether; cyclic ethers such as tetrahydrofuran and dioxane; alkanes; cycloalkanes; aromatic hydrocarbon solvents such as benzene, toluene, o-xylene, m-xylene, p-xylene; halogenated hydrocarbon solvents; acetonitrile; lactones such as butyrolactone, and valerolactones; ketones such as acetone, methyl ethyl ketone, methyl isobutyl ketone, cyclopentanone, and cyclohexanone; sulfones such as tetramethylene sulfone, 3-methylsulfolane, 2,4-dimethylsulfolane, butadiene sulfone, methyl sulfone, ethyl sulfone, propyl sulfone, butyl sulfone, methyl vinyl sulfone, 2-(methylsulfonyl) ethanol, and 2,2′-sulfonyldiethanol; sulfoxides such as dimethyl sulfoxide; cyclic carbonates such as propylene carbonate, ethylene carbonate and vinylene carbonate; carboxylic acid esters such as ethyl acetate, Methyl Cellosolve™ and methyl formate; and other solvents such as methylene chloride, nitromethane, acetonitrile, glycol sulfite and 1,2-dimethoxyethane (glyme), and combinations of such solvents. The present polymerization may also be conducted in accordance with known suspension, emulsion and precipitation polymerization processes.

Polymerizing may be conducted at a temperature of from −78 to 200° C., preferably from 0 to 160° C. and most preferably from 20 to 100° C. The reaction should be conducted for a length of time sufficient to convert at least 10% (preferably at least 50/a, more preferably at least 75% and most preferably at least 90%) of the monomer to polymer. Typically, the reaction time for complete cure will be from several minutes to 5 days, preferably from 30 minutes to 3 days, and most preferably from 1 to 24 hours.

Optionally the polymerizable composition comprises a “two-part” system in which the transition metal complex is in the first mixture, and the oxidizing agent, the photolabile reducing agent is generally in a second mixture. The polymerizable monomer may be part of the first and/or second mixture and is preferably in the first mixture. The two parts are combined, optionally coated on a substrate, and the redox reaction initiated by exposure to actinic radiation. In another embodiment, the polymerizable composition comprises a “two-part” system in which the transition metal complex, photolabile reducing agent and polymerizable monomer component is in the first mixture, and the oxidant is in the second mixture.

The combination of polymerizable composition and the redox initiator system may be irradiated with UV radiation to cleave or fragment the photolabile reducing agent, initiate the redox cycle and polymerize the polymerizable component(s). UV light sources can be of two types: 1) relatively low light intensity sources such as backlights which provide generally 10 mW/cm² or less (as measured in accordance with procedures approved by the United States National Institute of Standards and Technology as, for example, with a Uvimap® UM 365 L-S radiometer manufactured by Electronic Instrumentation & Technology, Inc., in Sterling, VA) over a wavelength range of 280 to 400 nanometers and 2) relatively high light intensity sources such as medium pressure mercury lamps which provide intensities generally greater than 10 mW/cm², preferably between 15 and 450 mW/cm². UV LEDs may also be used, such as a Clearstone UV LED lamp (Clearstone Technologies Inc.. Hopkins, MN 385 nm).

In some cases, the polymerizable composition is a single part composition. It was discovered that the photolabile reducing agent of Formula I can advantageously be incorporated into a single part composition that remains stable for days to weeks either at ambient temperature or when refrigerated (e.g., about 4° C.). The single part composition can be irradiated as described above to initiate polymerization.

The above-described compositions are coated on a substrate using conventional coating techniques modified as appropriate to the particular substrate. For example, these compositions can be applied to a variety of solid substrates by methods such as stencil printing, screen printing, roller coating, flow coating, dip coating, spin coating, spray coating, knife coating, and die coating. These various methods of coating allow the compositions to be placed on the substrate at variable thicknesses thus allowing a wider range of use of the compositions.

The polymerizable compositions may be coated upon a variety of flexible and inflexible substrates using conventional coating techniques to produce coated articles. Flexible substrates are defined herein as any material which is conventionally utilized as a tape backing or may be of any other flexible material. Examples include, but are not limited to, plastic films such as polypropylene, polyethylene, polyvinyl chloride, polyester (polyethylene terephthalate), polycarbonate, polymethyl(meth)acrylate (PMMA), cellulose acetate, cellulose triacetate, and ethyl cellulose. Foam backings may be used.

In some preferred embodiments, the substrate may be chosen so as to be transparent to the UV radiation used to initiate the redox cycle. The coated article may then be initiated through the thickness of the transparent substrate.

In some embodiments, the substrate is a release liner to form an adhesive article of the construction substrate/adhesive layer/release liner or release liner/adhesive/release liner. The adhesive layer may be cured, uncured or partially cured. Release liners typically have low affinity for the curable composition. Exemplary release liners can be prepared from paper (e.g., Kraft paper) or other types of polymeric material. Some release liners are coated with an outer layer of a release agent such as a silicone-containing material or a fluorocarbon-containing material. Release coating can be applied by solvent or solvent-free methods.

### Photolabile Reducing Agents

In a second aspect, the present disclosure provides a photolabile reducing agent. The photolabile reducing agent may be any photolabile reducing agent of Formula I according to the first aspect described in detail above. Examples of suitable synthesis methods for preparing the photolabile reducing agent are described in the Examples below.

### Exemplary Embodiments

In a first embodiment, the present disclosure provides a polymerizable composition. The polymerizable composition comprises a polymerizable component, and a redox initiation system comprising: a) a transition metal complex that participates in a redox cycle; b) an oxidizing agent; and c) a photolabile reducing agent of the Formula I:

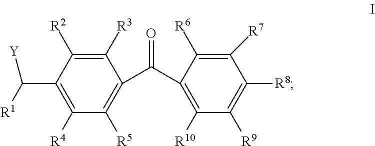

In Formula I, R¹ is of Formula II, Formula III, Formula IV, or Formula V; wherein Y is H or methyl; and wherein each of R², R³, R⁴, R⁵, R⁶, R⁷, R⁸, R⁹, and R¹⁰ is independently H, an alkyl, an alkoxy, or an aryl;

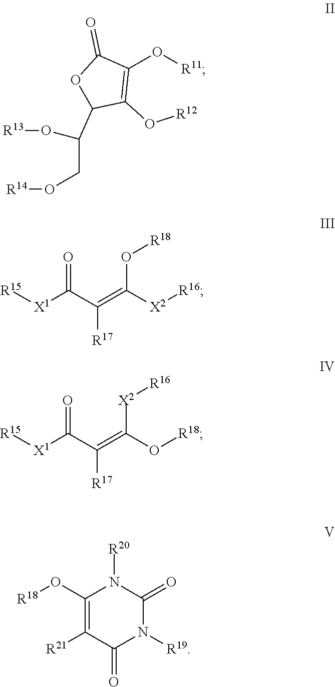

In Formulas II, III, IV, and V: R¹² is a covalent bond to Formula I at the R¹ position; R¹¹ is H, an alkyl, an aryl, or a covalent bond to a second group of Formula I at the R¹ position; each of R¹³ and R¹⁴ independently, in combination with the attached O, comprises an alcohol, an alkoxy, an aryloxy, an ester, an ether, a urethane, or a carbonate functional group, or taken together form a 5- or 6-membered ring; each of R¹⁵ and R¹⁶ is independently an optionally substituted 1-18C hydrocarbyl; R¹⁷ is H or an optionally substituted 1-18C hydrocarbyl, wherein R¹⁵+R¹⁶, or R¹⁵+R¹⁷, or R¹⁶+R¹⁷ are optionally taken together to form a 5- or 6-membered ring; R¹⁸ is a covalent bond to Formula I at the R¹ position; each of R¹⁹, R²⁰, and R¹² is H or an optionally substituted 1-18C hydrocarbyl; and each of X¹ and X² is independently a covalent bond, O, S, —N(R²²)—, —[C(R²²)₂]_{y=1,2,3}-, —CO— or —CO—O—; wherein R²² is H or a 1-18C alkyl.

In a second embodiment, the present disclosure provides a polymerizable composition according to the first embodiment, wherein each of R², R³, R⁴, R⁵, R⁶, R⁷, R⁸, R⁹, and R¹⁰ is H.

In a third embodiment, the present disclosure provides a polymerizable composition according to the first embodiment or the second embodiment, wherein the transition metal complex is of the formula [MLₚ]ⁿ⁺[A^{m-}]_{q}, wherein M is a transition metal selected from groups 5 to 11 in the Periodic Table of the Elements that participates in a redox cycle, L is a ligand, A^{m-} is an anion having a negative charge of m−; m and n are independently integers from 1 to 7; p is an integer from 1 to 9; and q is a whole number selected such that m·q=n.

In a fourth embodiment, the present disclosure provides a polymerizable composition according to any of the first through third embodiments, wherein M is selected from the group consisting of Cu, Fe, Ru, Cr, Mo, Pd, Ni, Pt, Mn, Rh, Re, Co, V, Au, Nb, and Ag.

In a fifth embodiment, the present disclosure provides a polymerizable composition according to the third embodiment or the fourth embodiment, wherein the transition metal complex is a first transition metal complex and the polymerizable composition further comprises a second transition metal complex, and wherein the M of the first transition metal complex is different than the M of the second transition metal complex.

In a sixth embodiment, the present disclosure provides a polymerizable composition according to any of the first through fifth embodiments, wherein the redox initiator system is present in the composition in an amount of from 0.05 to about 10 parts by weight, based on 100 parts by weight of the polymerizable component of the polymerizable composition.

In a seventh embodiment, the present disclosure provides a polymerizable composition according to any of the first through sixth embodiments, wherein the polymerizable component comprises at least one ethylenically unsaturated monomer.

In an eighth embodiment, the present disclosure provides a polymerizable composition according to any of the first through seventh embodiments, wherein the polymerizable component comprises at least one vinyl monomer or (meth)acrylate monomer or oligomer.

In a ninth embodiment, the present disclosure provides a polymerizable composition according to any of the first through eighth embodiments, wherein R¹ of Formula I is of Formula II.

In a tenth embodiment, the present disclosure provides a polymerizable composition according to any of the first through eighth embodiments, wherein R¹ of Formula I is of Formula III.

In an eleventh embodiment, the present disclosure provides a polymerizable composition according to any of the first through eighth embodiments, wherein R¹ of Formula I is of Formula IV.

In a twelfth embodiment, the present disclosure provides a polymerizable composition according to any of the first through eighth embodiments, wherein R¹ of Formula I is of Formula V.

In a thirteenth embodiment, the present disclosure provides a polymerizable composition according to any of the first through twelfth embodiments, wherein the oxidizing agent comprises at least one of an organic peroxide, an organic hydroperoxide, or an inorganic peroxide.

In a fourteenth embodiment, the present disclosure provides a polymerizable composition according to any of the first through thirteenth embodiments, further comprising a quaternary ammonium halide or a tertiary ammonium halide.

In a fifteenth embodiment, the present disclosure provides a photolabile reducing agent. the photolabile reducing agent is of the Formula I:

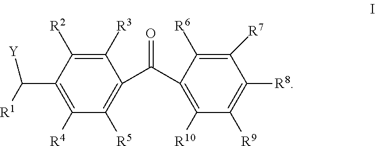

In Formula I, R¹ is of Formula II, Formula III, Formula IV, or Formula V; wherein Y is H or methyl; and wherein each of R², R³, R⁴, R⁵, R⁶, R⁷, R⁸, R⁹, and R¹⁰ is independently H, an alkyl, an alkoxy, or an aryl;

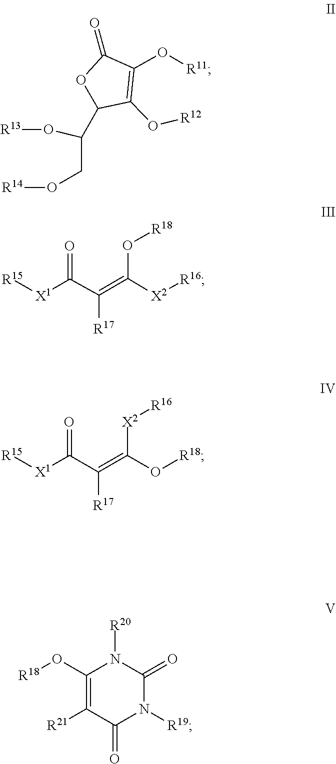

In Formulas II, III, IV, and V: R¹² is a covalent bond to Formula I at the R¹ position; R¹¹ is H, an alkyl, an aryl, or a covalent bond to a second group of Formula I at the R¹ position; each of R¹³ and R¹⁴ independently, in combination with the attached O, comprises an alcohol, an alkoxy, an aryloxy, an ester, an ether, a urethane, or a carbonate functional group, or taken together form a 5- or 6-membered ring; each of R¹⁵ and R¹⁶ is independently an optionally substituted 1-18C hydrocarbyl; R¹⁷ is H or an optionally substituted 1-18C hydrocarbyl, wherein R¹⁵+R¹⁶, or R¹⁵+R¹⁷, or R¹⁶+R¹⁷ are optionally taken together to form a 5- or 6-membered ring; R¹⁸ is a covalent bond to Formula I at the R¹ position; each of R⁹, R²⁰, and R¹ is H or an optionally substituted 1-18C hydrocarbyl; and each of X¹ and X² is independently a covalent bond, O, S, —N(R²²)—, —[C(R²²)₂]_{y=1,2,3}-, —CO— or —CO—O—; wherein R²² is H or a 1-18C alkyl.

In a sixteenth embodiment, the present disclosure provides a photolabile reducing agent according to the fifteenth embodiment, wherein each of R², R³, R⁴, R⁵, R⁶, R⁷, R⁸, R⁹, and R¹⁰ is H.

In a seventeenth embodiment, the present disclosure provides a photolabile reducing agent according to the fifteenth embodiment or the sixteenth embodiment, wherein R¹ of Formula I is of Formula II.

In an eighteenth embodiment, the present disclosure provides a photolabile reducing agent according to the fifteenth embodiment or the sixteenth embodiment, wherein R¹ of Formula I is of Formula III.

In a nineteenth embodiment, the present disclosure provides a photolabile reducing agent according to the fifteenth embodiment or the sixteenth embodiment, wherein R¹ of Formula I is of Formula IV.

In a twentieth embodiment, the present disclosure provides a photolabile reducing agent according to the fifteenth embodiment or the sixteenth embodiment, wherein R¹ of Formula I is of Formula V.

Advantages and embodiments of this invention are further illustrated by the following examples, but the particular materials and amounts thereof recited in these examples, as well as other conditions and details, should not be construed to unduly limit this invention. All parts and percentages are by weight unless otherwise indicated.

### EXAMPLES

Unless otherwise noted or apparent from the context, all parts, percentages, ratios, etc. in the Examples and the rest of the specification are by weight. Table 1 (below) lists materials used in the examples and their sources.

TABLE 1
Materials List

| DESIGNATION | DESCRIPTION | SOURCE |
|---|---|---|
| Acetone | Acetone | VWR International (Radnor, PA, |
| | | USA) |
| Ascorbyl Palmitate | Ascorbyl palmitate | Alfa Aesar (Ward Hill, MA, USA) |
| AIBN | 2,2′-Azobis(2-methylpropionitrile) | MilliporeSigma (Burlington, MA, |
| | | USA) |
| Benzene | Benzene | EMD Millipore Corp. (Billerica, |
| | | MA, USA) |
| Benzyl tri-n- | Benzyl tri-n-butylammonium | Alfa Aesar |
| butylammonium | chloride | |
| chloride | | |
| Benzyl | Benzyl triethylammonium chloride | Alfa Aesar |
| triethylammonium | | |
| chloride | | |
| BnMA | Benzyl methacrylate | TCI America Inc. (Portland, OR, |
| | | USA) |
| BYK-S782 | Resin additive | BYK USA Inc. (Wallingford, CT, |
| | | USA) |
| Cabosil | Cabosil TS-720 | Cabot Corp. (Billerica, MA, USA) |
| 10- | 10-Camphorsulfonic acid | MilliporeSigma |
| Camphorsulfonic | | |
| acid | | |
| CDCl₃ | Chloroform-d | Cambridge Isotope Laboratories, |
| | | Inc. (Andover, MA, USA) |
| Cs₂CO₃ | Cesium carbonate | Alfa Aesar |
| Cu naph | Copper (II) naphthenate, 6% in | Pfaltz & Bauer (Waterbury, CT, |
| | mineral spirits | USA) |
| CHP | Cumene hydroperoxide | MilliporeSigma |
| DMAP | 4-Dimethylamino pyridine | Alfa Aesar |
| d₆-DMSO | Dimethyl sulfoxide-d₆ | Cambridge Isotope Laboratories, |
| | | Inc. |
| 2,2- | 2,2-Dimethoxypropane | TCI Co. (Tokyo, Japan) |
| Dimethoxypropane | | |
| Diol6000 | Methacrylate crosslinker obtained | 3M Company St. (Paul, MN, |
| | from methacrylate functionalization | USA) |
| | of 6000 molecular weight polyether | |
| | (ethylene oxide/tetramethylene oxide | |
| | 1/3-4) diol, as described in | |
| | EP670341 | |
| Dioxane | 1,4-Dioxane | Alfa Aesar |
| DMSO | Dimethyl sulfoxide | VWR International |
| Dimethyl urea | Dimethyl urea | Alfa Aesar |
| ESS50F | HDPE fibers | Minifibers, Inc. (Johnson City, |
| | | TN, USA) |
| EtOAc | Ethyl acetate | VWR International |
| Hexanes | Hexanes | EMD Millipore Corp. |
| HEMA | 2-Hydroxyethyl methacrylate | TCI America Inc. (Portland, OR, |
| | | USA) |
| K₂CO₃ | Potassium carbonate | VWR International |
| KFlex | Benzoflex - 9-88 Plasticizer | Eastman Chemical Company |
| | | (Kingsport, TN, USA) |
| K₃PO₄ | Potassium phosphate tribasic | Alfa Aesar |
| L-Ascorbic acid | L-Ascorbic acid | Alfa Aesar |
| MEK | Methyl ethyl ketone | Avantor Performance Materials |
| | | (Center Valley, PA, USA) |
| 4-Methyl | 4-Methyl benzophenone | Oakwood Chemical (Estill, SC, |
| benzophenone | | USA) |
| 2-Methyl-1,3- | 2-Methyl-1,3-cyclohexanedione | Alfa Aesar |
| cyclohexanedione | | |
| MgSO₄ | Magnesium sulfate, anhydrous | EMD Millipore Corp. |
| N- | N-Bromosuccinimide | Alfa Aesar |
| Bromosuccinimide | | |
| NaCl | Sodium chloride | MilliporeSigma |
| NaHCO₃ | Sodium bicarbonate | VWR Chemicals (Solon, OH, |
| | | USA) |
| NaOH | Sodium hydroxide | MilliporeSigma |
| PAM200 | phosphate esters of polypropylene | Solvay Novacure (Cranbury, NJ, |
| | glycol monomethacrylate obtained as | USA) |
| | SIPOMER PAM 200 | |
| Phenylmalonic | Phenylmalonic acid | TCI Co. |
| acid | | |
| POCl₃ | Phosphorous (V) oxychloride | Alfa Aesar |
| Propylene | Propylene carbonate | Alfa Aesar |
| carbonate | | |
| PU3701 | Aromatic trifunctional methacrylate | Miwon Specialty Chemical Co., |
| | obtained as Miramer PU3701 | Ltd. (Anyang, South Korea) |
| SiO₂ | Silica gel 60, 0.040-0.063 mm | Alfa Aesar |
| SR348 | Ethoxylated (2) bisphenol A | Arkema (Exton, PA, USA) |
| | dimethacrylate obtained as SR348 | |
| SR540 | Ethoxylated (4) bisphenol A | Arkema |
| | dimethacrylate obtained as SR540 | |
| Succinic anhydride | Succinic anhydride | MilliporeSigma |
| H₂SO₄ | Sulfuric acid, concentrated | EMD Millipore Corp. |
| TBEC | Tert-Butylperoxy 2-ethylhexyl | MilliporeSigma |
| | carbonate | |
| THF | Tetrahydrofuran | EMD Millipore Corp. |
| Trifluoroacetic | Trifluoroacetic anhydride | Oakwood Chemical |
| anhydride | | |
| Toluene | Toluene | EMD Millipore Corp. |
| Triethylamine | Triethylamine | EMD Millipore Corp. |
| UDMA | Urethane dimethacrylate obtained as | Arkema |
| | CN1964 | |
| XT100 | MMA-butadiene-styrene (MBS) | Arkema |
| | core-shell impact modifier obtained | |
| | as CLEARSTRENGTH XT100 | |
| 5% AmCl | A solution of 5 weight percent | See sources for components above |
| | (wt. %) benzyl tri-n-butylammonium | |
| | chloride in HEMA | |

### General Procedures and Test Methods

### Preparation of Base and Accelerator Formulations

Base and accelerator formulations were prepared individually. Each formulation was prepared by combining all components into a polypropylene mixing cup (from FlackTek, Inc., Landrum, SC, USA). The cup was closed with a polypropylene lid and the mixture was high shear mixed at ambient temperature and pressure using a SPEEDMIXER (Hauschild SpeedMixer inc., Dallas, Texas, USA) for at least 60 seconds (s) at 2000 revolutions per minute (rpm).

### Preparation of Fourier Transform Infrared Spectroscopy (FTIR) Sample Sandwiches

The components of a given mixture were combined in a polypropylene mixing cup and blended in the SPEEDMIXER for 30 s at 2000 rpm. Directly after mixing, a large drop of formulation was sandwiched between two glass microscope slides: setup consists of a top slide (1 inch (in)×3 in (2.5 cm×7.6 cm), pre-cleaned, VWR 48300-025 from VWR, Radnor, PA, USA)+silicone rubber gasket (15 mil (0.4 mm) thick, 1 in×3 in (2.5 cm×7.6 cm)+bottom slide (2 in×3 in (5.1 cm×7.6 cm), pre-cleaned, VWR 48382-179), attached with small binder clips at top and bottom. The rubber gasket had a circle in the middle cut out to allow room for the formulation.

### Cure Monitoring by FTIR (Individual Spectrum)

A given FTIR sample sandwich was placed into a Nicolet IR iS50 spectrometer (Nicolet Thermo Fisher Scientific Inc., Waltham, MA, USA). Spectra were taken in a range of 4000-7000 cm⁻¹. These spectra were taken at specific times that are defined in each individual example. The spectra were analyzed for disappearance of the acrylate/methacrylate overtone peak measured from 6185-6145 cm⁻¹. This disappearance was translated into a % cure value.

### Cure Monitoring by FTIR (Series)

A given FTIR sample sandwich was placed into a Nicolet IR iS50 spectrometer (Nicolet Thermo Fisher Scientific Inc.). A series of spectra was collected in the range of 4000-7000 cm⁻¹. One spectrum was taken every 5 seconds for a total time defined in each specific example. This disappearance was translated into a % cure value. The series of spectra were analyzed for disappearance of the acrylate/methacrylate overtone peak measured from 6185-6145 cm⁻¹. This disappearance was translated into a % cure value at each point in the series.

Irradiation of Sandwich Sample with LX-400 in FTIR Spectrometer

A given IR Sandwich Sample was placed into a Nicolet IR iS50 spectrometer (Nicolet Thermo Fisher Scientific Inc.). The circular cutout in the sandwich that contains the resin was irradiated by an LX-400 UV LED light source (Excelitas Technologies Corporation, Waltham, MA). The UV light had a wavelength of 365 nm, the power was set to 1000, and the distance between the light source and the sample was 0.5 in (1.3 cm). In some cases, this irradiation was done while a series of IR spectra was in progress. The timing details of the irradiation are defined in each individual example.

### Overlap Shear (OLS) Testing

Aluminum substrates (1 inch×4 inches×0.064 inch, (2.5 cm×10 cm×0.16 cm)) to be tested were washed with methyl ethyl ketone, air dried for at least 10 minutes, then abraded with a Scotch abrasion pad (#7447 General Purpose Hand Pad, 3M Company, St. Paul, MN, USA). A freshly mixed example adhesive formulation was spread at 10 mil (0.25 millimeter (mm)) thick using a BYK-Gardner multiple clearance square applicator, 2″, 5-50 mils (Thomas Scientific, Swedesboro, NJ, USA) over the abraded portion of the substrate. The adhesive on the substrate was exposed to ultraviolet (UV) radiation as defined in the individual examples. A second abraded aluminum substrate was applied to the irradiated sample, thus closing the bond (bond area=0.5 inch×1 inch (1.3 cm×2.5 cm)). The bond was clamped with binder clips and allowed to sit at room temperature for 24 hours prior to testing. Dynamic overlap shear testing was performed at ambient temperature using an MTS Criterion C43 Tensile Tester (MTS Systems, Eden Prairie, MN, USA), specimens were loaded into the grips and the crosshead was operated at 0.2 inches per minute (0.5 cm/min), loading the specimen to failure. Specimens were created and tested in quadruplicate, with average and standard deviation values reported. Stress at break was recorded in units of pounds per square inch (psi) and converted to megapascals (MPa).

### PREPARATIVE EXAMPLES

### Preparative Example 1 (PE-1): 5,6-O-isopropylidene-L-ascorbic acid

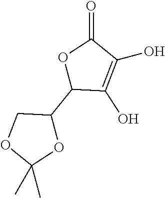

5,6-O-isopropylidene-L-ascorbic acid was prepared according to literature precedence (Bioorg. Med. Chem. 2003, 11, 827). To a suspension of L-ascorbic acid (20.0 grams (g), 114 millimoles (mmol)) in acetone (200 milliliters (mL)) was added 2,2-dimethoxypropane (20.4 g, 196 mmol) and 10-camphorsulfonic acid (1.32 g, 5.68 mmol). The resultant mixture was allowed to stir overnight at room temperature. To the resultant slurry was added approximately 0.6 g triethylamine. A portion of hexanes (approximately 50 mL) was added to the mixture, and the resultant white precipitate was collected via vacuum filtration, washing with additional hexanes. The material was dried under vacuum to afford 21.0 g of 5,6-O-isopropylidene-L-ascorbic acid (PE-1, 86% yield).

### Preparative Example 2 (PE-2): 4-bromomethyl benzophenone

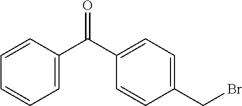 [SMILES: O=C(c1ccccc1)c1ccc(CBr)cc1]

4-methyl benzophenone (15.7 g, 80.0 mmol) and N-bromosuccinimide (15.7 g, 88.0 mmol, 1.1 equivalents (eq)) were dissolved in benzene (120 mL), and AIBN (0.64 grams, 4.0 mmol, 0.05 eq) was added. The resultant solution was heated to gentle reflux overnight. The following morning, the benzene was removed under reduced pressure, and the residue was dissolved in EtOAc (150 mL) and washed with saturated aqueous NaHCO₃ (2×75 mL) and saturated aqueous NaCl (1×75 mL). The organic layer was dried over MgSO₄, filtered, and concentrated to provide a white solid. This material was triturated with hexanes, then collected via filtration to afford 20.0 g of 4-bromomethyl benzophenone (PE-2) as a white solid (91% yield).

### Preparative Example 3 (PE-3): 4-hydroxymethyl benzophenone

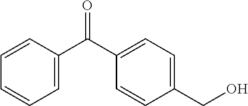 [SMILES: O=C(c1ccccc1)c1ccc(CO)cc1]

A mixture of PE-2 (2.76 g, 10.0 mmol) and K₂CO₃ (50.0 mmol, 6.91 grams) in 100 mL of 1:1 dioxane/H₂O was heated at reflux overnight. The majority of the dioxane was removed under reduced pressure, and the remaining mixture was extracted with EtOAc (2×75 mL). The combined organic layers were washed with saturated aqueous NaCl, dried over MgSO₄, and filtered. The material was then adsorbed onto silica gel and purified via suction filter column (SiO₂, ramp eluent from 7/1 to 3/1 hexanes/EtOAc). Product fractions were combined and concentrated to afford 1.90 g of 4-hydroxymethyl benzophenone (PE-3) as a yellow oil (90% yield).

### Preparative Example 4 (PE-4): 5-phenyl-1,3-dimethylbarbituric acid

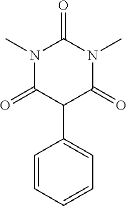 [SMILES: CN1C(=O)C(c2ccccc2)C(=O)N(C)C1=O]

A suspension of phenylmalonic acid (7.21 g, 40.0 mmol) and dimethyl urea (3.52 g, 40.0 mmol) in toluene (40 mL) was cooled in an ice bath. Trifluoroacetic anhydride (18.48 g, 88.0 mmol, 2.2 eq) was added via pipette. The reaction mixture quickly became a clear, very slightly pale yellow solution. The reaction mixture was slowly warmed to ambient temperature with stirring overnight. The following morning, the clear, yellow solution was extracted with 40 mL portions of aqueous 1 normal (N) NaOH. The first two portions were still acidic and were discarded. The third, fourth, and fifth portions were basic, ensuring complete removal of the desired product from the organic layer. The third, fourth, and fifth aqueous NaOH portions were combined, and dilute H₂SO₄ was added, causing the desired product to precipitate as a white solid. Once pH of <2 was reached, the product was collected via filtration, washing with additional portions of water. After drying, this provided the 5-phenyl-1,3-dimethylbarbituric acid (PE4, 6.70 g, 72% yield) as a white solid.

### Preparative Example 5 (PE-5): 6-chloro-5-phenyl-1,3-dimethyluracil

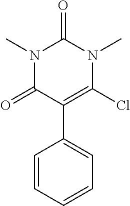 [SMILES: Cn1c(Cl)c(-c2ccccc2)c(=O)n(C)c1=O]

Benzyl triethylammonium chloride (45.6 g, 200 mmol) was added to a solution of PE-4 (23.2 g, 100 mmol) in phosphorous oxychloride (POCl₃, 100 mL), and the resultant mixture was heated at 50° C. overnight. The majority of the POCl₃ was then removed under reduced pressure, and the residue was dissolved in EtOAc (100 mL). Water (100 mL) was added to generate a biphasic mixture, and aqueous 3 N NaOH was then added until the aqueous layer had reached a pH of >8. The organic layer was then washed with saturated aqueous NaCl, dried over MgSO₄, filtered, and concentrated to provide 19.0 grams of 6-chloro-5-phenyl-1,3-dimethyluracil (PE-5, 76% yield) as a yellow solid.

### Preparative Examples 6 TO 17 (PE-6 TO PE-17): Adhesive Base Formulations

Adhesive base formulations PE-6 to PE-17 were made according to the General Procedure for Preparation of Base and Accelerator Formulations using the components listed in Tables 2 and 3.

TABLE 2
Components of Adhesive Base Formulations PE-6 to PE-12

| COMPONENT | PE-6 | PE-7 | PE-8 | PE-9 | PE-10 | PE-11 | PE-12 |
|---|---|---|---|---|---|---|---|
| HEMA, g | 3.40 | 2.90 | 2.40 | 1.90 | 3.20 | 2.25 | 2.10 |
| PAM200, g | 0.10 | 0.10 | 0.10 | 0.10 | 0.10 | 0.10 | 0.10 |
| XT100, g | 0.84 | 0.84 | 0.84 | 0.84 | 0.84 | 0.84 | 0.84 |
| Cabosil, g | 0.15 | 0.15 | 0.15 | 0.15 | 0.15 | 0.15 | 0.15 |
| Cu naph, g | 0.01 | 0.01 | 0.01 | 0.01 | 0.01 | 0.01 | 0.01 |
| 5% AmCl, g | | | | | 0.20 | 0.15 | 0.30 |
| UDMA, g | 0.50 | 1.00 | 1.50 | 2.00 | 0.50 | 1.50 | 1.50 |

TABLE 3
Components of Adhesive Base Formulations PE-13 to PE-17

| COMPONENT | PE-13 | PE-14 | PE-15 | PE-16 | PE-17 |
|---|---|---|---|---|---|
| HEMA, g | 2.40 | 2.40 | 2.40 | 2.40 | 4.36 |
| PAM200, g | 0.10 | 0.10 | 0.10 | 0.10 | 0.60 |
| XT100, g | 0.84 | 0.84 | 0.84 | 0.84 | 3.44 |
| Cabosil, g | 0.15 | 0.15 | 0.15 | 0.15 | 0.60 |
| Cu naph, g | 0.01 | 0.01 | 0.01 | 0.01 | 0.04 |
| UDMA, g | | | | | 6.00 |
| SR348, g | 1.50 | | | | |
| Diol6000, g | | 1.50 | | | |
| PU3701, g | | | 1.50 | | |
| SR540, g | | | | 1.50 | |
| BnMA, g | | | | | 4.36 |
| BYK-S782, g | | | | | 0.20 |
| ESS50F, g | | | | | 0.40 |

### Preparative Examples 18 TO 24 (PE-21 TO PE-24): Adhesive Accelerator Formulations

Adhesive accelerator formulations PE-18 to PE-24 were made according to the General Procedure for Preparation of Base and Accelerator Formulations using the components listed in Table 4.

TABLE 4
Components of Adhesive Accelerator Formulations PE-18 to PE-24

| COMPONENT | PE-18 | PE-19 | PE-20 | PE-21 | PE-22 | PE-23 | PE-24 |
|---|---|---|---|---|---|---|---|
| EX-1, g | 0.48 | | | | | | |
| EX-2, g | | | 0.23 | | | | |
| EX-3, g | | 0.72 | | | | | |
| EX-4, g | | | | | 0.48 | | 0.48 |
| EX-5, g | | | | | | 0.48 | |
| Kflex, g | 1.56 | 1.32 | 1.72 | 1.70 | 1.56 | 1.56 | 1.08 |
| TBEC, g | 0.30 | 0.30 | | 0.30 | 0.30 | 0.30 | 0.30 |
| CHP, g | | | 0.15 | | | | |
| Propylene | | | | | | | 0.51 |
| carbonate, g | | | | | | | |
| XT100, g | 0.36 | 0.36 | 0.60 | 0.70 | 0.36 | 0.36 | 0.36 |
| Cabosil, g | 0.30 | 0.30 | 0.30 | 0.30 | 0.30 | 0.30 | 0.27 |

### EXAMPLES

### Example 1 (EX-1): Benzophenone-blocked ascorbic acid acetonide

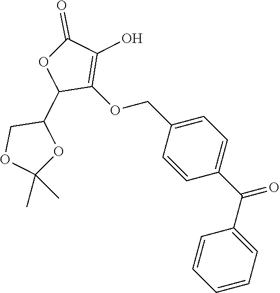 [SMILES: CC1(C)OCC(C2OC(=O)C(O)=C2OCc2ccc(C(=O)c3ccccc3)cc2)O1]

K₂CO₃ (2.76 g, 20.0 mmol) was added to a solution of PE-1 (4.32 g, 20.0 mmol) and PE-2 (2.76 g, 10.0 mmol) in 100 mL of 1:1 THF/DMSO in a round-bottom flask equipped with a stir bar. The resultant mixture was allowed to stir at room temperature overnight. The following morning, aqueous 1 N HCl (150 mL) was added. The mixture was extracted with EtOAc (3×75 mL). The combined organic layers were washed with water (3×75 mL) and saturated aqueous NaCl (1×75 mL), then dried over MgSO₄, filtered, and adsorbed onto silica gel. Purification via suction filter column (SiO₂), ramping eluent from 4/1 to 3/1 hexanes/EtOAc, provided 2.90 grams of EX-1 as a pale-yellow oil (71% yield).

### Example 2 (EX-2): Benzophenone-blocked ascorbic acid

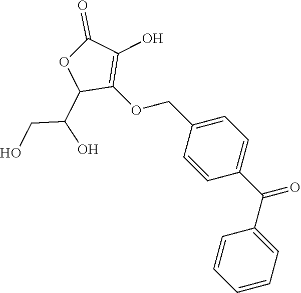 [SMILES: O=C1OC(C(O)CO)C(OCc2ccc(C(=O)c3ccccc3)cc2)=C1O]

A solution of EX-1 (2.05 g, 5.0 mmol) in 100 mL of 1:1 THF/1 N aqueous HCl was made and allowed to stir overnight. The following morning, water (100 mL) was added to the mixture, which was then extracted with EtOAc (3×75 mL). The combined organic layers were washed with saturated aqueous NaCl (1×75 mL) then dried over MgSO₄, filtered, and adsorbed onto silica gel. Purification via suction filter column (SiO₂, 1/1 hexanes/EtOAc eluent) provided 1.62 g of EX-2 as a pale-yellow oil (87% yield).

### Example 3 (EX-3): Benzophenone-blocked ascorbyl palmitate

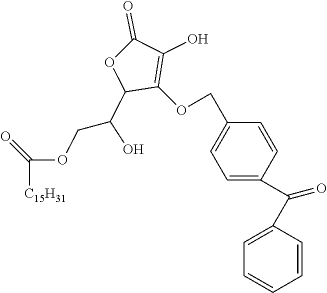

Potassium carbonate (2.76 g, 20.0 mmol) was added to a solution of ascorbyl palmitate (8.29 g, 20.0 mmol) in 80 mL of 1:1 THF/DMSO in a round-bottom flask equipped with a stir bar. The resultant mixture was allowed to stir at ambient temperature for 1 hour (h). A solution of PE-2 (5.50 g, 20.0 mmol) in 40 mL of 1:1 THF/DMSO was added dropwise via addition funnel over 30 minutes. The resultant mixture was allowed to stir at ambient temperature overnight. The following morning, volatile solvents were removed under reduced pressure. Water (200 mL) was added to the residue and the mixture was extracted with EtOAc (3×75 mL). The combined organic layers were washed with water (3×100 mL) and saturated aqueous NaCl (1×75 mL), then dried over MgSO₄, filtered, and concentrated to an orange oil. This material was adsorbed onto silica gel and purified via suction filter column (SiO₂, ramp eluent from 7/1 to 3/1 hexane/EtOAc) to provide 4.60 grams of EX-3 as a highly viscous yellow oil (38% yield).

### Example 4 (EX-4): Benzophenone-blocked 5-phenyl-1,3-dimethylbarbituric acid

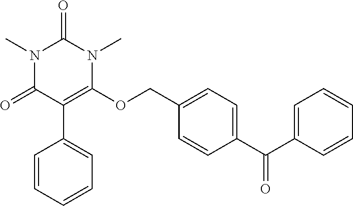 [SMILES: Cn1c(OCc2ccc(C(=O)c3ccccc3)cc2)c(-c2ccccc2)c(=O)n(C)c1=O]

Cesium carbonate (2.90 g, 8.91 mmol) was added to a solution of PE-3 (1.89 g, 8.91 mmol) and PE-5 (1.49 g, 5.94 mmol) in methyl ethyl ketone (30 mL) in a round-bottom flask equipped with a stir bar. The resultant mixture was allowed to stir at room temperature over 72 h. Water (100 mL) was added, and the resultant mixture was extracted with EtOAc (3×75 mL). The combined organic layers were washed with saturated aqueous NaCl, dried over MgSO₄, filtered, and concentrated to provide approximately 3.1 grams of a yellow oil. This material was dissolved in acetone (30 mL), then succinic anhydride (1.00 g, 10.0 mmol), potassium phosphate tribasic (4.25 g, 20.0 mmol) and DMAP (approx. 0.2 g) were added. The resultant mixture was allowed to stir at room temperature for 40 hours. Water (200 mL) was added to the mixture which was then extracted with EtOAc (3×75 mL). The combined organic layers were washed with saturated aqueous NaHCO₃ and saturated aqueous NaCl, dried over MgSO₄, filtered, and adsorbed onto silica gel. Purification via suction filter column (SiO₂, ramp eluent from 6/1 to 3/2 hexane/EtOAc) affords 1.0 g of EX-4 (39% yield) as a pale yellow crystalline solid.

### Example 5 (EX-5): Benzophenone-blocked 2-methyl-1,3-cyclohexanedione

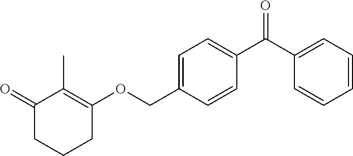 [SMILES: CC1=C(OCc2ccc(C(=O)c3ccccc3)cc2)CCCC1=O]

To a solution of PE-3 (2.74 grams, 12.9 mmol) in 100 mL toluene in a round-bottom flask equipped with a stir bar was added 2-methyl-1,3-cyclohexanedione (1.79 g, 14.2 mmol, 1.1 eq), followed by 10-camphorsulfonic acid (0.15 g, 0.65 mmol, 0.05 eq). A Dean-Stark trap was placed on the reaction vessel, and the reaction mixture was heated at reflux overnight. The following morning, the toluene was removed via rotovap, and the residue was dissolved in EtOAc (100 mL) and washed with saturated aqueous NaHCO₃ (3×50 mL), water (50 mL) and saturated aqueous NaCl (50 mL). The organic layer was dried over MgSO₄, filtered, and concentrated to afford 2.20 g of EX-5 (53% yield) as a yellow oil.

### Examples 6 to 20 (EX-6 to EX-20) and Comparative Example 1 (CE-1): Cure Monitoring of Mixed Adhesive Formulations

Mixed adhesive formulations EX-6 to EX-20 and CE-1 were prepared by combining 2.0 g of the given base formulation (from Tables 2 and 3) with 0.2 g of the given accelerator formulation (from Table 4) in a polypropylene mixing cup (from FlackTek, Inc., Landrum, SC, USA). In each case the cup was closed with a polypropylene lid and the mixture was high shear mixed at ambient temperature and pressure using a SPEEDMIXER (Hauschild SpeedMixer inc., Dallas, Texas, USA) for 60 s at 2000 rpm.

FTIR sample sandwiches of EX-6 to EX-20 were independently prepared according to the General Procedure for Preparation of FTIR Sample Sandwiches. In some cases, more than one sample sandwich was made for a given formulation so that different UV irradiation times could be explored. Measurement of curing (monomer conversion) was carried out for each sample sandwich according to the General Procedure for Cure Monitoring by FTIR (Individual Spectrum). In each case, measurement of the monomer conversion was started at time (t)=0 minutes, and UV irradiation (duration listed in Table 5) was applied starting at time=2 minutes. Note that the UV irradiation was carried out within the FTIR spectrometer according to the General Procedure for Irradiation of Sandwich Sample with LX-400 in FTIR Spectrometer. Note also that an irradiation time of 0 s indicates that no UV irradiation was applied to the sample sandwich.

For each sandwich, Table 5 lists the recorded % monomer conversion at time=1 minute (prior to irradiation), time=3 minutes (within 1 minute of completing irradiation, if any), time=30 minutes, and either time=24 hours or time=4 days (d).

TABLE 5
Results of Cure Monitoring Experiments on Mixed Adhesive Formulations

| | | | | % MONOMER CONVERSION | | | | |
|---|---|---|---|---|---|---|---|---|
| EXAMPLE | BASE | ACCELERATOR | IRRADIATION, s | t = 1 min | t = 3 min | t = 30 min | t = 24 h | t = 4 d |
| EX-6 | PE-6 | PE-18 | 15 | 0.2 | 21.2 | 38.8 | 81.3 | |
| EX-7 | PE-7 | PE-18 | 15 | 0.0 | 27.1 | 38.7 | 73.6 | |
| EX-8 | PE-8 | PE-18 | 15 | 0.5 | 32.4 | 43.5 | 76.4 | |
| EX-9 | PE-9 | PE-18 | 15 | 0.0 | 36.0 | 48.5 | 76.4 | |
| EX-10 | PE-10 | PE-18 | 0 | 0.0 | 0.2 | 0.5 | | 4.3 |
| | | | 10 | 0.1 | 27.0 | 40.6 | | 88.4 |
| | | | 15 | 0.0 | 34.2 | 44.7 | | 84.5 |
| | | | 30 | 0.0 | 46.8 | 56.3 | | 92.4 |
| EX-11 | PE-8 | PE-19 | 15 | 0.0 | 23.6 | 31.7 | 52.9 | |
| EX-12 | PE-11 | PE-19 | 15 | 0.1 | 25.6 | 36.0 | 50.1 | |
| EX-13 | PE-12 | PE-19 | 15 | 0.6 | 25.3 | 33.1 | 55.2 | |
| EX-14 | PE-8 | PE-20 | 0 | 0.1 | 0.1 | 0.3 | 2.8 | |
| | | | 15 | 0.0 | 41.8 | 52.9 | 81.4 | |
| EX-15 | PE-13 | PE-20 | 0 | 0.0 | 0.2 | 0.6 | 3.3 | |
| | | | 15 | 0.5 | 27.0 | 36.5 | 65.6 | |
| EX-16 | PE-14 | PE-20 | 0 | 0.3 | 0.4 | 1.1 | 5.4 | |
| | | | 5 | 0.8 | 25.6 | 57.1 | 85.2 | |
| | | | 15 | 0.3 | 69.2 | 88.8 | 93.0 | |
| EX-17 | PE-15 | PE-20 | 0 | 0.0 | 0.2 | 0.6 | 1.9 | |
| | | | 5 | 0.0 | 18.1 | 41.6 | 67.7 | |
| | | | 15 | 0.3 | 70.7 | 93.1 | 94.3 | |
| CE-1 | PE-8 | PE-21 | 15 | 0.0 | 0.3 | 0.9 | 5.3 | |
| EX-18 | PE-8 | PE-23 | 0 | 0.0 | 0.2 | 0.4 | 3.3 | |
| | | | 15 | 0.7 | 19.3 | 26.2 | 46.9 | |
| EX-19 | PE-8 | PE-22 | 0 | 0.1 | 0.4 | 0.4 | 5.0 | |
| | | | 5 | 0.5 | 19.8 | 49.0 | 84.0 | |
| | | | 10 | 0.4 | 47.1 | 66.8 | 86.6 | |
| | | | 15 | 0.0 | 71.1 | 78.9 | 88.4 | |
| EX-20 | PE-16 | PE-22 | 0 | 0.1 | 0.3 | 0.4 | 4.2 | |
| | | | 5 | 0.4 | 13.9 | 39.3 | 69.9 | |
| | | | 10 | 0.2 | 27.6 | 61.0 | 86.4 | |
| | | | 15 | 0.3 | 47.8 | 70.4 | 87.8 | |
| | | | 20 | 0.0 | 67.6 | 80.3 | 90.5 | |

### Example 21 (EX-21): Overlap ShearTesting on a Mixed Adhesive Formulation

The mixed adhesive formulation EX-21 was prepared by combining 4.0 g of PE-17 with 0.4 g of PE-24 in a polypropylene mixing cup (from FlackTek, Inc., Landrum, SC, USA). The cup was closed with a polypropylene lid and the mixture was high-shear mixed at ambient temperature and pressure using a SPEEDMIXER (Hauschild SpeedMixer inc., Dallas, Texas, USA) for 60 s at 2000 rpm. The freshly mixed formulation was then tested according to the General Procedure for Overlap Shear Testing. Several specimens were made and subjected to different irradiation conditions. The UV source was the D Bulb of aFusion UV Processor (Heraeus, Hanau, Germany). The total amount of UV-A radiation applied to the specimens was measured on a POWERPUCK II (EIT, Sterling, VA, USA) and reported on Table 6. The results of the overlap shear testing are also reported on Table 6.

TABLE 6
Overlap Shear Testing Results

| | IRRADIATION, | OLS STRENGTH | OLS STRENGTH |
|---|---|---|---|
| EXAMPLE | J/cm² | (AVERAGE), psi (MPa) | (STD. DEV.), psi (MPa) |
| EX-21 | 0* | 1 (0.0) | — |
| | 1 | 1193 (8.2) | 163 (1.1) |
| | 2 | 1264 (8.7) | 107 (0.7) |
| | 3 | 1480 (10.2) | 169 (1.2) |

*Only one specimen was run with no irradiation, as it was clear that no curing of the adhesive occurred.

Foreseeable modifications and alterations of this invention will be apparent to those skilled in the art without departing from the scope and spirit of this invention. This invention should not be restricted to the embodiments that are set forth in this application for illustrative purposes.

1. A polymerizable composition comprising a polymerizable component, and a redox initiation system comprising:
a transition metal complex that participates in a redox cycle;
an oxidizing agent; and
a photolabile reducing agent of the Formula I: 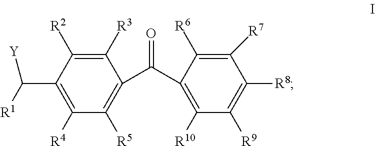
wherein R¹ is of Formula II, Formula III, Formula IV, or Formula V; wherein Y is H or methyl; and wherein each of R², R³, R⁴, R⁵, R⁶, R⁷, R⁸, R⁹, and R¹⁰ is independently H, an alkyl, an alkoxy, or an aryl; 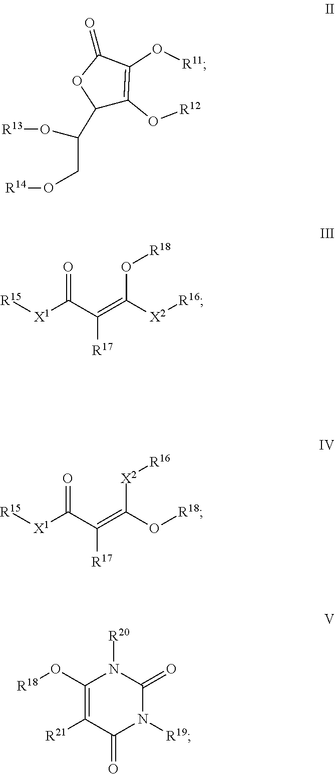
wherein:
R¹² is a covalent bond to Formula I at the R¹ position;
R¹¹ is H, an alkyl, an aryl, or a covalent bond to a second group of Formula I at the R¹ position;
each of R¹³ and R¹⁴ independently, in combination with the attached O, comprises an alcohol, an alkoxy, an aryloxy, an ester, an ether, a urethane, or a carbonate functional group, or taken together form a 5- or 6-membered ring;
each of R¹⁵ and R¹⁶ is independently an optionally substituted 1-18C hydrocarbyl;
R¹⁷ is H or an optionally substituted 1-18C hydrocarbyl, wherein R¹⁵+R¹⁶, or R¹⁵+R¹⁷, or R¹⁶+R¹⁷ are optionally taken together to form a 5- or 6-membered ring;
R¹⁸ is a covalent bond to Formula I at the R¹ position;
each of R¹⁹, R²⁰, and R²¹ is H or an optionally substituted 1-18C hydrocarbyl; and
each of X¹ and X² is independently a covalent bond, O, S, —N(R²²)—, —[C(R²²)₂]_{y=1,2,3}—, —CO— or —CO—O—; wherein R² is H or a 1-18C alkyl.

2. The polymerizable composition of claim 1, wherein each of R², R³, R⁴, R⁵, R⁶, R⁷, R⁸, R⁹, and R¹⁰ is H.

3. The polymerizable composition of claim 1, wherein the transition metal complex is of the formula [MLₚ]ⁿ⁺[A^{m-}]_{q}, wherein M is a transition metal selected from groups 5 to 11 in the Periodic Table of the Elements that participates in a redox cycle, L is a ligand, A^{m-} is an anion having a negative charge of m−; m and n are independently integers from 1 to 7; p is an integer from 1 to 9; and q is a whole number selected such that m·q=n.

4. The polymerizable composition of claim 1, wherein M is selected from the group consisting of Cu, Fe, Ru, Cr, Mo, Pd, Ni, Pt, Mn, Rh, Re, Co, V, Au, Nb, and Ag.

5. The polymerizable composition of claim 3, wherein the transition metal complex is a first transition metal complex and the polymerizable composition further comprises a second transition metal complex, and wherein the M of the first transition metal complex is different than the M of the second transition metal complex.

6. The polymerizable composition of claim 1, wherein the redox initiator system is present in the composition in an amount of from 0.05 to about 10 parts by weight, based on 100 parts by weight of the polymerizable component of the polymerizable composition.

7. The polymerizable composition of claim 1, wherein the polymerizable component comprises at least one ethylenically unsaturated monomer.

8. The polymerizable composition of claim 1, wherein the polymerizable component comprises at least one vinyl monomer or (meth)acrylate monomer or oligomer.

9. The polymerizable composition of claim 1, wherein R¹ of Formula I is of Formula II.

10. The polymerizable composition of claim 1, wherein R¹ of Formula I is of Formula III.

11. The polymerizable composition of claim 1, wherein R¹ of Formula I is of Formula IV.

12. The polymerizable composition of claim 1, wherein R¹ of Formula I is of Formula V.

13. The polymerizable composition of claim 1, wherein the oxidizing agent comprises at least one of an organic peroxide, an organic hydroperoxide, or an inorganic peroxide.

14. The polymerizable composition of claim 1, further comprising a quaternary ammonium halide or a tertiary ammonium halide.

15. A photolabile reducing agent of the Formula I: 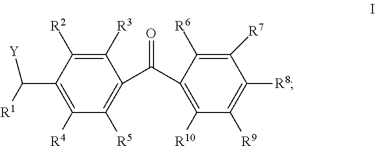
wherein R¹ is of Formula II, Formula III, Formula IV, or Formula V; wherein Y is H or methyl; and wherein each of R², R³, R⁴, R⁵, R⁶, R⁷, R⁸, R⁹, and R¹⁰ is independently H, an alkyl, an alkoxy, or an aryl; 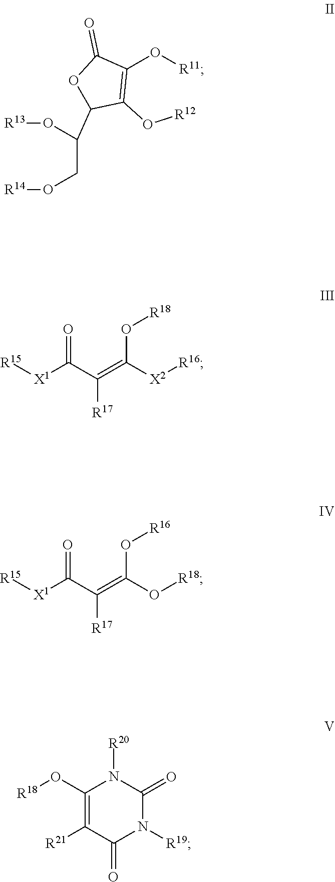
wherein:
R¹² is a covalent bond to Formula I at the R¹ position;
R¹¹ is H, an alkyl, an aryl, or a covalent bond to a second group of Formula I at the R¹ position;
each of R¹³ and R¹⁴ independently, in combination with the attached O, comprises an alcohol, an alkoxy, an aryloxy, an ester, an ether, a urethane, or a carbonate functional group, or taken together form a 5- or 6-membered ring;
each of R¹⁵ and R¹⁶ is independently an optionally substituted 1-18C hydrocarbyl;
R¹⁷ is H or an optionally substituted 1-18C hydrocarbyl, wherein R¹⁵+R¹⁶, or R¹⁵+R¹⁷, or R¹⁶+R¹⁷ are optionally taken together to form a 5- or 6-membered ring;
R¹⁸ is a covalent bond to Formula I at the R¹ position;
each of R¹⁹, R²⁰, and R²¹ is H or an optionally substituted 1-18C hydrocarbyl;
each of X¹ and X² is independently a covalent bond, O, S, —N(R²²)—, —[C(R²²)₂]_{y=1,2,3}-, —CO— or —CO—O—; wherein R²² is H or a 1-18C alkyl.

16. The photolabile reducing agent of claim 15, wherein each of R², R³, R⁴, R⁵, R⁶, R⁷, R⁸, R⁹, and R¹⁰ is H.

17. The photolabile reducing agent of claim 15, wherein R¹ of Formula I is of Formula II.

18. The photolabile reducing agent of claim 15, wherein R¹ of Formula I is of Formula III.

19. The photolabile reducing agent of claim 15, wherein R¹ of Formula I is of Formula IV.

20. The photolabile reducing agent of claim 15, wherein R¹ of Formula I is of Formula V.
